(19) United States
(12) Patent Application Publication
(10) Pub. No.: US 2003/0114936 A1
(43) Pub. Date: Jun. 19, 2003

(54) Complex three-dimensional composite scaffold resistant to delimination

(75) Inventors: Jill K. Sherwood, Edison, NJ (US); Donald Monkhouse, Radnor, PA (US); Christopher M. Gaylo, Princeton Junction, NJ (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Therics, Inc.

(21) Appl. No.: 10/207,531

(22) Filed: Jul. 29, 2002

Related U.S. Application Data
(63) Continuation-in-part of application No. 09/416,346, filed on Oct. 12, 1999, now Pat. No. 6,454,811.
(60) Provisional application No. 60/103,853, filed on Oct. 12, 1998.

(51) Int. Cl. A61F 2/28

(52) U.S. Cl. 623/23.58; 623/23.76; 623/923; 435/402

(57) ABSTRACT

The devices disclosed herein are composite implantable devices having a gradient of one or more of the following: materials, macroarchitecture, microarchitecture, or mechanical properties, which can be used to select or promote attachment of specific cell types on and in the devices prior to and/or after implantation. In preferred embodiments, the implants include complex three-dimensional structure, including curved regions and saddle-shaped areas. In various embodiments, the gradient forms a transition zone in the device from a region composed of materials or having properties best suited for one type of tissue to a region composed of materials or having properties suited for a different type of tissue. Methods to improve these devices for use in repair or replacement of cartilage and/or bone have been developed, which specifically address 1) the selection of the appropriate polymeric material for the cartilage region, 2) mechanical testing of the bone region including the effect of porosity and polymer/calcium phosphate ratio, and 3) prevention of delamination in the transition region.

This application is a continuation-in-part application of U.S. Ser. No. 09/416,346 filed Oct. 12, 1999.

### FIELD OF THE INVENTION

The invention relates generally to implantable devices characterized by gradients of materials, architecture, and/or properties for tissue regeneration, made using solid free-form fabrication technology, which can be combined with computer-aided design.

### BACKGROUND OF THE INVENTION

Over 16 million people in the U.S. suffer from severe joint pain and related dysfunction, such as loss of motion, as a result of injury or osteoarthritis. In particular, loss of function of the knees can severely impact mobility and thus the patient's quality of life. The biological basis of joint problems is the deterioration of articular cartilage, which covers the bone at the joint surface and performs many complex functions. Articular cartilage is composed of hyaline cartilage which has unique properties, such as viscoelastic deformation, that allow it to absorb shock, distribute loads, and facilitate stable motion. Self-repair of hyaline cartilage is limited and the tissue that forms is usually a combination of hyaline and fibrocartilage, which does not perform as well as hyaline cartilage and can degrade over time.

Current treatments for articular defects have limited success in that they are deficient in long-term repair or have unacceptable side effects. Autograft procedures, such as Mosaicplasty and Osteochondral Autolograft Transfer System (OATS), remove an osteochondral plug from a non-load bearing area and graft it into the defect site. Despite the recent successes this procedure has seen in repairing cartilage lesions, it requires additional time and money to acquire the donor tissue and results in donor site morbidity and pain. CARTICEL®, a procedure consisting of injecting cells under a periosteal flap, has also had limited success; however, the procedure lacks inter-patient consistency with some patients maintaining little relief months or years later, and the surgical procedure is technically challenging. Abrasion arthroscopy, subchondral bone drilling and microfracture typically result in fibrocartilage filling the defect site. Allogenic transplantation of osteochondral grafts has had clinical success, but supply is limited and has a risk of infection.

Each of the currently used repair modalities has severe limitations, and the outcome is generally regarded as inadequate. Tissue engineering of cartilage has great potential in providing the appropriate replacement tissue with features necessary for a successful repair of cartilage to occur. While there has been success in growing cartilage in vitro, success in vivo requires reliable fixation into the joint defect and integration with the subchondral bone. Ultimately, for defects in articular locations with substantial curvature, the tissue engineered constructs should also have appropriate topography.

Cartilage is an avascular tissue composed of 5-10% by weight of living cells. There are three major types of cartilage in the body: hyaline, fibrocartilage, and elastic cartilage. Hyaline cartilage covers the epiphyses of the bone and, in synovial joints, lies within a fluid filled capsule. Fibrocartilage composes the intervertebral discs separating the vertebrae of the spinal columns. Elastic cartilage is present in areas requiring extreme resilience, such as the tip of the nose. Cartilage is formed by and contains cells called chondrocytes. The extracellular matrix of hyaline cartilage contains closely packed Type II collagen fibers and proteoglycans including hyaluronate and glycoaminoglycans in a chondroitin sulfate matrix. Chondrocytes receive nutrients and dispose of wastes by diffusion through the matrix and are believed to have limited mobility or ability to divide and regenerate damaged tissue. Chondrocytes normally produce anti-angiogenesis factors. However, when large areas of cartilage are damaged, overgrowth by fibroblasts and neovascularization of the area may result in the formation of scar tissue or callus instead of articular cartilage. A subsequent ingrowth of bone forming cells may result in calcium deposition in these areas, causing further deformation of the local area.

The interface between bone and cartilage is therefore the interface between a vascularized and avascular tissue as well as mineralized (ossified) and nonminerilized collagen matrices. Traumatic injury, as well as such conditions as osteoarthritis and aging, often result in damage to the articular cartilage, which may also involve damage to the underlying bone. Therefore, there is a need for a method of treatment which meets the disparate needs of both tissue types and allows or encourages the healing process to progress towards restoration of both types of tissues at the same site.

Clinical use of grafts of living tissue have recently moved from direct implantation of freshly harvested fully formed tissue, e.g. skin grafts or organ transplants, to strategies involving seeding of cells on matrices which will regenerate or encourage the regeneration of local structures. For complex and weight bearing hard tissues, there is an additional need to provide mechanical support of the existing structure by replacement or substitution of the hard tissue for at least some of the healing period. Thus, the device must serve as a scaffold of specific architecture which will encourage the migration, residence and proliferation of specific cell types as well as provide mechanical and structural support during healing. In the case of devices for regeneration of articular (hyaline) cartilage, it is important that the device be completely resorbable, as residual material may compromise the surface integrity (smoothness) and overall strength and resilience of the regenerated tissue.

In order to encourage cellular attachment and growth, the overall porosity of the device is important. Additionally, the individual pore diameter or size is an important factor in determining the ability of cells to migrate into, colonize, and differentiate while in the device (Martin, R B et al. Biomaterials, 14: 341, 1993). For skeletal tissues, bone and cartilage, guided support to reproduce the correct geometry and shape of the tissue is thought to be important. It is generally agreed that pore sizes of above 150 μm and preferably larger (Hulbert, et al., 1970; Klawitter, J. J, 1970; Piecuch, 1982; and Dennis, et al., 1992) and porosity greater than 50% are necessary for cell invasion of the carrier by bone forming cells. It has been further accepted that a tissue regenerating scaffold must be highly porous, greater than 50% and more preferably more than 90%, in order to facilitate cartilage formation.

It is well documented that the physiological processes of wound healing and tissue regeneration proceed sequentially with multiple cell types and that cellular factors play a role. For example, thrombi are formed and removed by blood elements, which are components of cascades regulating both coagulation and clot lysis. Cells which are not terminally differentiated, such as fibroblasts, migrate into the thrombus and lay down collagen fibers. Angiogenic cells are recruited by chemotactic factors, derived from circulating precursors or released from cells, to form vascular tissue. Finally, cells differentiate to form specialized tissue. The concept of adding exogenous natural or synthetic factors in order to hasten the healing process is also an area of intense exploration, and numerous growth factors, such as cytokines, angiogenic factors, and transforming factors, have been isolated, purified, sequenced, and cloned. Determining the correct sequence and concentration in which to release one or multiple factors is another area of research in the field of tissue engineering.

Several attempts to address some of the above problems of tissue regeneration in a graft or implantable device have been disclosed. U.S. Pat. No. 5,270,300 describes a method for treating defects or lesions in cartilage or bone which provides a matrix, possibly composed of collagen, with pores large enough to allow cell population, and which further contains growth factors or other factors (e.g. angiogenesis factors) appropriate for the type of tissue desired to be regenerated. U.S. Pat. No. 5,270,300 specifically teaches the use of TGF-beta in the matrix solution as a proliferation and chemotactic agent at a lower concentration and at a subsequent release of the same factor at a higher concentration to induce differentiation of cartilage repair cells. In the case of a defect in adjoining bone and cartilage, a membrane is secured between the bone-regenerating matrix and the cartilage-regenerating matrix to prevent blood vessel penetration from one site to the other site.

U.S. Pat. No. 5,607,474 to Athanasiou et al. describes a molded carrier device comprising two bioerodible polymeric materials having dissimilar mechanical properties arranged proximate to each other for the purpose of being placed in the body adjoining two dissimilar types of tissues. Each polymeric material has a variable degree of porosity or pore sizes into which tissue cells can enter and adhere. The two components of the device are fabricated separately and, e.g., bonded together in a mold. Other features, such as larger passages for cell access, can be mechanically placed in the device.

U.S. Pat. No. 5,514,378 attempts to address some of the requirements of providing a highly porous biocompatible and bioerodible device using a method of forming membranes from a polymer and particle solution. The pores are created by removing the particles, achieved by dissolving and leaching them away in a solvent, such as water, which does not dissolve the polymer, thereby leaving a porous membrane. The polymer must be soluble in a non-aqueous solvent and is limited to synthetic polymers. Once the membrane is created it may be cast into the desired shape. It is envisioned that such membranes could also be laminated together to form a three-dimensional shape.

It has been further recognized that not only the morphology of such devices but the materials of which they are composed will contribute to the regeneration processes as well as the mechanical strength of the device. For example, some materials are osteogenic and stimulate the growth of bone forming cells; some materials are osteoconductive, encouraging bone-forming cell migration and incorporation; and some are osteoinductive, inducing the differentiation of mesenchymal stem cells into osteoblasts. Materials which have been found to be osteogenic usually contain a natural or synthetic source of calcium phosphate. Osteoinductive materials include molecules derived from members of the transforming growth factor-beta (TGF-beta) gene superfamily including: bone morphogenetic proteins (BMPs) and insulin-like growth factors (IGFs).

U.S. Pat. No. 5,626,861 teaches a composite material for use as bone graft or implant composed of biodegradable, biocompatible polymer and a particulate calcium phosphate, hydroxyapatite. The calcium phosphate ceramic was added in order to increase the mechanical strength over the polymer alone and to provide a “bone bonding” material. The material is produced in such a manner as to provide irregular pores between 100 and 250 microns in size.

An approach to making suitable devices using three-dimensional printing is described in PCT/US99/23732 by Massachusetts Institute of Technology and Therics. The methods described in this application overcome many of the problems with prior art devices, providing for structural elements, structure gradients as well as gradients of porosity and composition to control seeding and ingrowth, and complete biodegradability.

It is an object to provide improved three dimensional printing methods and device designs for repair and replacement of cartilage.

### SUMMARY OF THE INVENTION

The devices disclosed herein are composite implantable devices having a gradient of one or more of the following: materials, macroarchitecture, microarchitecture, or mechanical properties, which can be used to select or promote attachment of specific cell types on and in the devices prior to and/or after implantation. In preferred embodiments, the implants include complex three-dimensional structure, including curved regions and saddle-shaped areas. In various embodiments, the gradient forms a transition zone in the device from a region composed of materials or having properties best suited for one type of tissue to a region composed of materials or having properties suited for a different type of tissue. Methods to improve these devices for use in repair or replacement of cartilage and/or bone have been developed, which specifically address 1) the selection of the appropriate polymeric material for the cartilage region, 2) mechanical testing of the bone region including the effect of porosity and polymer/calcium phosphate ratio, and 3) prevention of delamination in the transition region.

The devices are made in a continuous process that imparts structural integrity as well as a unique gradient of materials in the architecture. The gradient may relate to the materials, the macroarchitecture, the microarchitecture, the mechanical properties of the device, or several of these together. The devices disclosed herein typically are made using solid free form processes, especially three-dimensional printing process (3DP™). Other types of solid free-form fabrication (SFF) methods include stereo-lithography (SLA), selective laser sintering (SLS), ballistic particle manufacturing (BPM), and fusion deposition modeling (FDM). The device can be manufactured in a single continuous process such that the transition from one form of tissue regeneration scaffold to the other form of tissue regeneration scaffold has no “seams” and is less subject to differential swelling once the device is implanted into physiological fluid.

The resulting device is a fully resorbable synthetic scaffold, containing a cartilage-appropriate region and a bone-appropriate region, in a cell-scaffold-based tissue engineering approach to repair articular defects. Scaffolds are built one thin layer at a time, which allows for the production of devices having almost arbitrary spatial distribution of composition and geometric features, and provides the capability to fabricate devices with biologically and anatomically relevant features. The primary features of these scaffolds can include: 1) a highly porous cartilage region to facilitate seeding chondrocytes selectively in this region, 2) staggered channels in the cartilage region to promote homogeneous seeding throughout the 2-mm thickness of the region, 3) a cloverleaf bone region to promote bone ingrowth for fixation and integration while maintaining necessary mechanical characteristics, and 4) a transition region with a gradient of materials and pore structure to prevent delamination. Autologous chondrocytes that have been expanded in culture from a small biopsy or expanded allogenic chondrocytes that have been extensively tested for diseases can then be seeded onto the top portion of the scaffold. The seeded scaffold can then be cultured in vitro until adequate tissue formation has occurred and can then be implanted into the cartilage defect site.

### BRIEF DESCRIPTION OF THE DRAWINGS

FIG. 1 is a schematic of a laminated process in which a thin layer of powder is spread and then bound together in desired areas with a liquid binder.

FIG. 2 is a line drawing of bone showing the articular cartilage surfaces.

FIGS. 3a and 3b are illustrations of the construction of a complex three dimensional scaffold for forming a bone and cartilaginous composite implant.

FIGS. 4a, 4b and 4c are perspective views of the structures formed using the layering process shown schematically in FIGS. 3a and 3b to produce implants ultimately yielding bone and cartilaginous surfaces as shown in FIG. 2. FIG. 4a shows the assembled individual regions, separated from each other.

FIG. 5 is a graph of biochemical results of TheriForm™ scaffolds created with polymers 1-7 and cultured statically with dermal fibroblasts for 4 weeks. DNA and MTT values were significantly greater for polymer 4 (p<0.05, one-way ANOVA with Tukey post-hoc testing). Bars represent means±standard deviations for n=3, except for polymer 4 (n=2) and the DNA results for polymer 7 (n=2).

FIG. 6 is a graph of the amount of shrinkage of scaffolds after leaching for 48 hours.

FIG. 7 is a graph of the biochemical results for TheriForm™ osteochondral scaffolds that were seeded with OAC cells by a top or rotational seeding method and cultured statically for 4 weeks. The top seeding method resulted in greater number of cells and S-GAG content in the scaffolds (p<0.001). Collagen content was not statistically different for the two seeding methods and was most likely due to the large standard deviation of the rotational seeded samples. Bars represent means±standard deviations for n=3.

### DETAILED DESCRIPTION OF THE INVENTION

The advantages afforded to the manufacture of a three-dimensional device with unconventional microstructures and macroarchitecture are applied to the construction of complex alloplasts or partial allografts designed for tissue regeneration at a physiological junction between two types of supporting connective tissue. More specifically, the device is engineered in such a way as to allow and encourage growth of both osteogenic cells and chondrocytes. The overall shape of the device is such that the device functions to allow the continued flow of dissolved nutrients in biological or biocompatible fluids through and around the device, thus minimizing the possibility of pressure differential across the device being formed by gas, fluid or temperature gradients. The device may function is this regard while inserted in a physiological site requiring tissue support as well as tissue regeneration and thereby allow fluid flow to and from the areas of tissue damage and desired regeneration. The device may also be used in an extracorporeal device prior to placement in the body for purposes of cell seeding. This property is a function of the macroarchitecture or overall shape of the device. It is a further object of the invention that the device contains geometry, pores, and fluid communication channels which are conducive to cell migration, attachment, growth, and differentiation. In this way, the device functions to facilitate the regeneration of the complex supporting tissue interfaces which are characteristic of, for example, the cartilage coated surface of a long bone at the synovial interface.

In a preferred embodiment of the device resorbable or non-resorbable materials may be positioned in various portions of the device during the manufacturing process. The materials selected and so positioned will be selected from those materials known to be osteoconductive in one area of the device and those known to be permissive to chondrocyte growth and maturation in another part of the device. In yet a most preferred embodiment of the device, growth-stimulating factors may be deposited thereon or therein so as to be released in concert with the needs for growth and differentiation of the cell types involved.

In a preferred embodiment, the device is in the form of an insert with a first portion designed to support cartilage healing and regeneration, and a second portion designed to anchor in and support bone regeneration for use to treat osteochondral defects. More particularly, the device may be fabricated in a continuous process as a single part in which three regions, distinct in intent, design, and composition are present: 1) a cartilage portion, 2) a bone portion and 3) a transition zone adjacent to and connecting both the cartilage and bone portions. The cartilage portion is about 90% porous composed of synthetic polyester polymers containing staggered macro-channels of about 250 microns in diameter. The bone portion is from 25 to 55% porous and generally composed of both synthetic polymer and osteoconductive material in a shape permissive of fluid and gas flow at the outer edge of the device while maintaining contact with the host tissues.

The transition zone, which is apposed to both the cartilage and the bone portions, forms a gradient in porosity from close to that of the bone or more dense portion to close to that of the cartilage or least dense portion and may include variation of ratio of the polyester polymers and other materials found in both of the other portions also in gradient fashion. The transition zone moreover may have a shape gradient or have a region which has an outer shape like the bone portion near the bone portion and a region with an outer shape that is substantially round or similar to the cartilage portion in the region nearest the cartilage portion. The device so manufactured is not susceptible to delamination of the bone portion from the cartilage portion caused by differential swelling of the polymeric materials or other properties, such as the hygroscopic nature of, or osmotic pressure generated by the placement of dry materials in a fluid filled cavity or other fluid containing site in the body.

I. Three-dimensional Printing: A Solid Free-Form Fabrication Method

Solid free-form fabrication methods are used to manufacture devices for tissue regeneration and for seeding and implanting cells to form organ and structural components, which can additionally provide controlled release of bioactive agents. SFF methods can be used to selectively control composition within the build plane by varying the composition of printed material. The SFF methods can be adapted for use with a variety of polymeric, inorganic and composite materials to create structures with defined compositions, strengths, and densities, using computer aided design (CAD). This means that unconventional microstructures, such as those with complicated porous networks or unusual composition gradients, can be designed at a CAD terminal and built through an SFF process such as 3DP.

A. Methods of Manufacture using 3DP

3DP uses a process of spreading powder and depositing binder onto the powder bed. Three-dimensional printing is described by Sachs, et al., “CAD-Casting: Direct Fabrication of Ceramic Shells and Cores by Three-dimensional Printing: Manufacturing Review 5 (2), 117-126 (1992) and U.S. Pat. No. 5,204,055, the teachings of which are incorporated herein. Suitable apparatuses include both those with a continuous jet printhead and a drop-on-demand (DOD) printhead. 3DP can be used to create a porous bioerodible matrix for use as a medical device as taught in U.S. Pat. Nos. 5,490,962 and 5,518,680 the teachings of which are incorporated herein by reference.

A continuous jet head provides for a fluid that is pressure driven through a small orifice. Droplets naturally break off at a frequency that is a function of the fluid's properties and the orifice diameter. Initial prototype components and devices were built using a single jet head. Multiple jet heads are preferred. A microvalve DOD printhead utilizes individual solenoid valves that run at frequencies up to 1.2 kHz. Fluid is also pressure driven through these valves, and a small orifice is downstream of the valves to ensure accurate and repeatable droplet size. Piezoelectric DOD printheads use the action of a piezoelectric element to squeeze a drop of fluid through an orifice.

Both raster and vector apparatuses can be used. A raster apparatus provides that the printhead goes back and forth across the bed with motion in only one axis at any given time during printing. A vector apparatus similar to an x-y printer is capable of moving in two directions simultaneously during printing. 3DP is used to create a solid object by printing a binder onto selected areas of sequentially deposited layers of powder or particulates. In the following description, the terms “powder” and “particulates” are used interchangeably. Each layer may be created by spreading a thin layer of powder over the surface of a powder bed. In one embodiment, a moveable powder piston is located within a cylinder, with a powered roller to deliver dispensed powder to a receiving platform located adjacent to the powder feeder mechanism.

Operation consists of raising the feed piston a predetermined amount for each increment of powder delivery. The roller then sweeps across the surface of the powder feeder cylinder and deposits it as a thin layer across the receiving platform immediately adjacent to the powder feeder. The powder feeding piston is then lowered as the roller is brought back to the home position, to prevent any back delivery of powder.

The powder piston and cylinder arrangement can also consist of multiple piston/cylinders located in a common housing, which could be used to dispense multiple powders in the following sequence:

1. Line up the first desired powder cylinder with the rolling/delivery mechanism;

2. Increment the movable position piston up to deliver an incremental amount of powder;

3. Activate roller to move powder to receiving platform;

4. Lower the powder piston driving mechanism;

5. Laterally slide the powder feeder housing such that the next desired powder cylinder is lined up with the delivery mechanism;

6. Repeat steps 2, 3, 4 and 5; and

7. Continue for as many different powders and/or powder layers as required.

This method of powder feeding can be controlled manually or be fully automated. Cross contamination of different powders is minimized since each powder is contained in its own separate cylinder. One of the advantages to this method is that only one piston raising/lowering mechanism is required for operation, regardless of the number of powder cylinders. By raising the powder for delivery rather than dropping it from above, problems associated with gravity based delivery systems such as “ratholing”, incomplete feed screw filling/emptying and “dusting” with the use of fine powders is eliminated or minimized since only enough energy is introduced to move the powder up an incremental amount. The powder feeder housing, with its multiple cylinders and pistons, can also be designed as a removable assembly, which would minimize changeover times from one powder system to another.

The powder bed is supported by a piston which descends upon powder spreading and printing of each layer (or, conversely, the ink jets and spreader are raised after printing of each layer and the bed remains stationary). Instructions for each layer are derived directly from a computer-aided design (CAD) representation of the component. The area to be printed is obtained by computing the area of intersection between the desired plane and the CAD representation of the object. The individual sliced segments or layers are joined to form the three-dimensional structure. The unbound powder supports temporarily unconnected portions of the component as the structure is built but is removed after completion of printing.

The 3DP process steps are generally: Powder is rolled from a feeder source in stage I with a powder spreader onto a surface of a build bed. The thickness of the spread layer is varied as a function of the type of dosage form being produced. Generally, the thickness of the layer can vary from about 100 to about 500 microns, and more typically from 100 to about 200 microns. The printhead then deposits the binder (fluid) onto the powder layer and the build piston is lowered one layer distance. Powder is again rolled onto the build bed and the process is repeated until the dosage forms are completed. The droplet size of the fluid is from about 50 to about 500 microns in diameter and more typically greater than 80 microns. Servomotors are used to drive the various actions of the apparatus.

In another embodiment the powder layer can be deposited by dispensing a slurry or suspension which comprises the powder particles that will make up the layer, as described elsewhere herein.

Construction of a 3DP component can be viewed as the knitting together of structural elements that result from printing individual binder droplets into a powder bed. These elements are called microstructural primitives. The dimensions of the primitives determine the length scale over which the microstructure can be changed. Thus, the smallest region over which the concentration of bioactive agent can be varied has dimensions near that of individual droplet primitives. Droplet primitives have dimensions that are very similar to the width of line primitives formed by consecutive printing of droplets along a single line in the powder bed. The dimensions of the line primitive depend on the powder particle dimension and the amount of binder printed per unit line length. A line primitive of 500 micron width is produced if an inkjet depositing 1.1 cc/min of methylene chloride is made to raster at 8″/sec over the surface of a polycaprolactone (PCL) powder bed with 45-75 micron particle size. Higher printhead velocities and smaller particle size produce finer lines. The dimensions of the primitive seem to scale with that calculated on the assumption that the liquid binder or solvent needs to fill the pores of the region in the powder which forms the primitive.

Finer feature size is also achieved by printing polymer solutions rather than pure solvents. For example, a 10 wt. % PCL solution in chloroform produces 200 micron lines under the same conditions as above. The higher solution viscosity slows the migration of solvent away from the center of the primitive.

While the layers become hardened or at least partially hardened as each of the layers is laid down, once the desired final part configuration is achieved and the layering process is complete, in some applications it may be desirable that the form and its contents be heated or cured at a suitably selected temperature to further promote binding of the powder particles. In the case of matrices for implantable devices built from biocompatible materials, whether or not further curing is required, the loose unbonded powder particles may be removed using a suitable technique such as ultrasonic cleaning, to leave a finished device.

The solvent drying rate is an important variable in the production of polymer parts by 3DP. Very rapid drying of the solvent tends to cause warping of the printed component. Much, if not all, of the warping can be eliminated by choosing a solvent with a low vapor pressure. Thus, PCL parts prepared by printing chloroform have nearly undetectable amounts of warpage, while large parts made with methylene chloride exhibit significant warpage. It is often convenient to combine solvents to achieve minimal warping and adequate bonding between the particles. Thus, an aggressive solvent can be mixed in small proportions with a solvent with lower vapor pressure.

Significant amounts of matter can be deposited in selected regions of a component on a 100 micron scale by printing solid dispersions or solid precursors through the ink-jet printheads. Hundreds of jets can be incorporated into the process. The large number of individually controlled jets makes high rate 3DP construction possible.

Erodible devices are one of the simplest medical devices that can be constructed. These types of devices can be used in an oral or implantable form depending on the desired purpose and whether delivery of a specific bioactive agent is also desired. They differ in the materials used in the device construction, various physical parameters such as moldability and strength, and the time period over which the device erodes and bioactive agent is delivered. Lessons learned from the examples of individual erodible implants in terms of fabrication methods, behavior of the materials, and performance of these devices have been valuable in the design for the composite devices and the application of three-dimensional printing to their fabrication.

Manipulation of the printing parameters and powder characteristics allow the design and fabrication of macroarchitecture, microarchitecture, and internal and surface characteristics. “Macroarchitecture” is used herein to mean the overall shape of the device, which is on the order of millimeters to centimeters in dimension and with defined shape. The term “microarchitectural features” is used herein to mean the internal structure that is preconceived and built into the device. Fine features, such as tortuous interconnected pores and surface patterning are properties of the materials, processing, and finishing, but are not necessarily placed by design or by the three-dimensional printing process.

A bone replacement part designed to assure mechanical strength, density, and weight similar to that of bone logically may be assumed to require the appearance of cancellous bone in both internal and external structure. However, the healing process occurs in several stages and bone formation requires, in some cases, that cellular precursors undergo migration and differentiation before new bone is formed. Thus, the objective of a bone tissue or cartilage tissue healing device is not to imitate the configuration of the final tissue structure but rather to encourage and enhance the natural tissue formation process while contributing mechanical strength in the area to be regenerated.

The devices described herein can be manufactured with a gradient of materials or material mixtures. Using a gradient of materials allows the physical properties of the resulting structures to change gradually thereby mitigating large discontinuities which can lead to disruption of or performance failure by the device. Such physical properties of the materials include thermal expansion coefficient, elasticity, and swelling.

Macroarchitectural Design

The composite device is produced as a single part and is of an overall shape that when placed in the body will compress slightly while allowing structural features for fluid movement within and without the device to be maintained, with channels and pores, suitable for implantation in the body at an interface between two types of tissues. The bone region of the composite device is specifically designed to address several functions. One of these is to encourage the migration of the blood and marrow-bourne tissue forming elements around and through the device, to maximize the surface-area-to-volume ratio in order to promote bone ingrowth, and to maximize compressive and torsional strength in order to provide the mechanical integrity needed to withstand the force of implantation. Minimization of material without sacrificing integrity of the device was considered desirable whenever possible in order to decrease the cost of goods required in production as well as to minimize the introduction of foreign substances into the body which could potentially evoke an immune response and which releases degradation by-products. Designs contemplated for the bone portion of the composite device were analyzed on the basis of selected criteria including compressive strength, surface area available for cell adhesion, and ease of fabrication. Other criteria such as the ability to fabricate the device using masking rather than computer controlled printing were also considered for initial ease of prototype production.

Microarchitecture: Large Channels and Walls

Channels bounded by walls and consisting of substantially straight passageways of defined width, length, and orientation are a microarchitectural feature of the devices described herein. Staggered channels extending through the device and offset by 90° in different layers of the device are one particularly preferred embodiment. Staggering the channel and walls increases the strength of the device relative to a straight through channel design. The width of the channels can range from about 150 to 500 microns, with 250 microns preferred, in order to maximize the surface area available for cell seeding without compromising structural integrity or homogeneity of tissue formation.

In addition, the channels facilitate the transport of nutrient to the cells and removal of cellular by-products and polymer degradation by-products which all may occur whether the device is colonized by cells before or after implantation in the body. The unique macroscopic staggered channels are designed to allow chondrocytes to contact the device throughout the thickness of the device not only superficially. This is important due to the limited migration capacity of the chondrocytes; the migration distances of this cell type being less than about 2 mm. Thus, when the device is seeded extracorporeally, the chondrocytes may be placed directly into the center of the device.

Features: Porosity, Pore Size, and Surfaces

The porosity of a device will control the flow of nutrients to the colonizing cells as well as the surface area available for cellular attachment. Studies have shown that pores of a minimum diameter of 60 microns or greater are required for angiogenesis in highly vascularized tissue, such as bone. It is already known in the art that the porosity of the devices fabricated from powders or synthetic polymers or polymers and inorganic particles can be manipulated by incorporating “sacrificial” materials, such as sodium chloride, into the material. U.S. Pat. No. 5,514,378 teaches methods of dispersing salt particles in a biocompatible polymer solution, evaporating the polymer solvent and leaching the salt from the formed composite to create a porous membrane.

Fabrication of structures with designed pore or channel structures is a challenging task even with additive manufacturing processes such as 3DP. Structures with radial or vertical channels of hundreds of microns in diameter can be fabricated; however, the formation of narrower and tortuous internal structures is best effected by the use of a sacrificial material. One common practice in the construction of tissue engineering matrices is the use of mixtures of water soluble particulates (sodium chloride) with non-water soluble polymers dissolved in a solvent to fabricate specimens. The salt particles can be leached out of the device with water to reveal a porous structure. While this technique is useful in fabricating a network of pores, control of pore architecture is lost.

The microarchitectural feature of porosity was varied between the two tissue specific regions of the device. In the region designed specifically to enhance cartilage regeneration, the porosity was maximized (≧90%) to promote cell attachment and proliferation and allow space for formation of extracellular matrix. Highly porous structures have a high surface-to-volume ratio. The surface area maximizes available sites for cell attachment while minimizing the amount of material used. Minimizing material, besides allowing space for living components and promoting homogeneous formation of tissue, also minimizes the non-living foreign material which can cause immune response and produces potentially detrimental degradation by-products.

In the region of the device designed specifically to be implanted in bone, the device was less porous in order to provide for more mechanical strength and to discourage attachment of chondrocytes. The materials selected for this region are slowly degrading bioresorbable materials with an initially large pore size created by leaching out salt particles of 125 microns or greater.

A gradient of porosities is provided in the fabrication process design. In the three-dimensional printing process the final porosity gradient is achieved by altering the salt content of the powder bed in successive layers.

Surface finish of the devices of the invention is governed by the physical characteristics of the materials used as well as the build parameters. These factors include particle size, powder packing, surface characteristics of the particles and printed binder (i.e. contact angle), exit velocity of the binder jet, binder saturation, layer height, and line spacing. Interaction of the binder liquid with the powder surface, in particular, can be controlled carefully to minimize surface roughness. In a case where the binder becomes wicked out in a large area, the feature size control may be difficult, resulting in a rough surface.

The microporosity includes the interstitial spaces between bound or unbound particles. Microporosity is the porosity between individual joined powder particles. Macrochannels or other macro features are of a size scale or a large enough number of powder particles such that the unbound powder particles can be removed. The macroporosity or macrostructure may have long, approximately one-dimensional channels or holes that are empty or have reduced packing fraction on a small-size scale to foster the in-growth of natural bone.

The pore size and other feature geometry is designed to be conducive to in-growth of natural bone. The powder particles may be of aspect ratio reasonably close to spherical or equiaxial, or, alternatively, at least some fraction of the particles may be of somewhat more elongated geometry. The term “particles” is used herein to refer to all of these shapes. In the case of matrices in which the particles are joined directly to each other, the particles may be made of one or more ceramic or other inorganic substances. Examples of ceramics or other inorganic substances resembling substances found in natural bone are hydroxyapatite, tricalcium phosphate, and other calcium phosphates and compounds containing calcium and phosphorus. The particles may be polymer(s).

The matrix may have an overall exterior shape that includes geometric complexity. For example, the overall exterior shape may include undercuts, recesses, interior voids, and the like, provided that the undercuts, recesses, interior voids, and the like have access to the space outside the matrix. The matrix may be shaped appropriately so as to replace a particular bone or bones or segments of bones or spaces between bones or voids within bones. The matrix may be dimensioned and shaped uniquely for a particular patient prior to the start of surgery. Alternatively, the matrix could be simple overall shapes such as blocks, which are intended to be shaped by a surgeon during a surgical procedure. The matrix may be tightly fitting with respect to a defect in a bone. To aid fit, the matrix may be tapered or beveled or include some other interlocking feature.

The partially joined particles may form a three-dimensionally interconnected network. The space not occupied by the partially joined particles, may also form a three-dimensionally interconnected network that may interlock with the network formed by the partially joined particles. The space is referred to herein as the pores or porosity. Porosity may be characterized by the porosity fraction or void fraction, which is the fraction of the overall volume that is not occupied by particles or other solid material.

For an individual particle, an equivalent particle diameter can be defined as the diameter of a sphere having volume equal to that of a particle, and diameters of various particles may be averaged to give an average particle diameter of a collection of particles.

Pore size may involve a distribution of pore size. Pore size may be characterized by a pore size distribution which may be measured by mercury porosimetry and which may be presented as a graph of what fraction of the total pore volume is present in pores of a given size or size range, as a function of pore size. There may be one or more peaks in the pore size distribution, and each pore size which is at a peak may be considered to be a statistical mode for pore size, in terms of the fraction of the total pore volume which is contained by a given pore size or pore size interval.

In some embodiments, the matrix may have a designed internal geometric architecture comprising microstructure and macrostructure in the form of interstitial porosity, open holes, passageways or channels of size scale such that the smallest dimension of the hole passageway or channel is approximately equal to or larger than the diameter of the particle used. At least some part of the interconnected porosity, holes, passageways or channels has access to the space outside the matrix.

In one embodiment, the macrostructure includes holes or passageways or channels that may each have a cross-section that is substantially constant. In an alternative embodiment, the cross-section of the holes, passageways, channels or other macrostructural features may be variable. These holes, passageways or channels may be relatively long in one dimension in comparison to their other two dimensions. As illustrated below, the macrostructure provides paths or branches for in-growth of natural bone, cartilage or other tissue. Such holes or passageways or channels need not be straight; they can be curved, have changes of direction, have varying cross-section, and can branch to form other passageways or channels or holes or can intersect other passageways or channels or holes. Macrostructure channels may range from 2 to 2000 microns and typically range from 200 to 700 microns in size. The minimum cross-sectional dimension of a macro-channel is approximately the cross-sectional dimension of a primitive. The dimensions of the macrostructure channels may for example be 1 mm to 1.6 mm in each of the two dimensions in a cross-section perpendicular to the longest direction of the macrostructure. The matrix may have one surface which is parallel to the plane of the horizontal channels and which is essentially continuous, containing no macroscopic holes or channels through it.

In three-dimensional printing, a layer of powder is deposited such as by roller spreading or by slurry deposition. Examples of the powder substance are described herein. After the powder layer has been deposited, a binder liquid is deposited onto the powder layer in selected places so as to bind powder particles to each other and to already-solidified regions. The binder liquid may be dispensed in the form of successive discrete drops, a continuous jet, or other form. Binding may occur either due to deposition of an additional solid substance by the binder liquid, or due to dissolution of the powder particles or of a substance mixed in with the powder particles by the binder liquid, followed by resolidification. Following the printing of the binder liquid onto a particular layer, another layer of powder is deposited and the process is repeated for successive layers until the desired three-dimensional object is created. Unbound powder supports bound regions until the matrix is sufficiently dry, and then the unbound powder is removed. Another suitable method that could be used to deposit layers of powder is slurry deposition.

The liquid thus deposited in a given pass binds powder particles together so as to form in the powder bed a line of bound material that has dimensions of bound material in a cross-section perpendicular to the dispenser's direction of motion. This structure of bound powder particles may be referred to as a primitive. The cross-sectional dimension or line width of the primitive is related in part to the diameter of the drops if the liquid is dispensed by the dispenser in the form of discrete drops, or to the diameter of the jet if the liquid is deposited as a jet, and also is related to other variables such as the speed of motion of the printhead. The cross-sectional dimension of the primitive is useful in setting other parameters for printing. For printing of multiple adjacent lines, the line-to-line spacing may be selected in relation to the width of the primitive printed line. Typically the thickness of the deposited powder layer may be selected in relation to the dimension of the primitive printed line. Typical drop diameters may be in the tens of microns, or, for less-demanding applications, hundreds of microns. Typical primitive dimensions may be somewhat larger than the drop diameter. Printing is also described by a quantity called the saturation parameter. Parameters which influence printing may include flow rate of binder liquid, drop size, drop-to-drop spacing, line-to-line spacing, layer thickness, powder packing fraction, etc., and may be summarized as a quantity called the saturation parameter. If printing is performed with discrete drops, each drop is associated with a voxel (unit volume) of powder that may be considered to have the shape of a rectangular prism.

The ratio of the dispensed droplet volume to the empty volume in the voxel is the saturation parameter. The illustrated voxel has dimensions delta x, delta y and delta z, and has a powder packing fraction pf. The printhead fast axis speed and dispense interval may be given by V and delta T with the relation that (delta x)=V*(delta t). The drop volume may be represented by Vd. In this situation, the available empty volume in the voxel is given by (1−pf)*(delta x)*(delta y)*(delta z). The saturation parameter is given by Vd/(1−pf)*(delta x)*(delta y)*(delta z)).

A macrostructure such as a macro-channel may be made by printing bound regions so as to define a region of unbound powder by surrounding it with bound regions from all but at least one direction. A macrochannel may have a minimum dimension which is approximately the size of one primitive. Typically, in three-dimensional printing, if complete or nearly complete line-to-line and layer-to-layer binding is desired without excessive spreading of liquid, a saturation parameter approximately or slightly less than unity is used, for printing performed at room temperature.

A binder substance is a substance that is capable of binding powder particles to each other and to other solid regions. It may be absent from the finished matrix. An example of a binder substance is poly acrylic acid (PAA), which can be contained in an aqueous solution. Other examples are other soluble polymers and in general any substance which is soluble in a liquid. It is also possible, in the case where powder particles are polymers, to use a binder liquid which is itself a solvent for the solid, which will effect partial fusion of particles to each other by partial dissolution of particles followed by resolidification, without leaving any additional substance in the article. An example is PLGA particles with chloroform as a binder liquid.

Following the completion of three-dimensional printing and allowing sufficient time for the liquid in the binder liquid to evaporate, the printed matrix may be removed from the powder bed and unbound powder may be separated from it. This may be done by a simple process such as gentle shaking or brushing and may be further aided by techniques such as sonication such as are known in the art. At this point, the particles that are bound together may be held together by the binder substance, which may have solidified so as to surround or partially surround particles. Adjustment of the saturation parameter from one region of a matrix to another, using a given dispenser, may be achieved by adjusting any of the variables which together make up the saturation parameter. This may be achieved by adjusting the amount of dispensed liquid per-unit distance traveled along the principal direction of motion. In raster printing this may be adjusted by adjusting either the speed of the printhead or the timing of commands for drop ejection. For example, without adjusting the printhead speed, drops may be ejected at longer intervals of space or time in some regions, and at shorter intervals of space or time in other regions. For example, a doubling of saturation parameter may be achieved by dispensing in some print regions a drop at every location of a scheduled pattern, and by dispensing in other print regions a drop only at every second location in that same pattern. Some dispensing technologies, such as piezoelectric, may permit continuous (within some range) variation of the local saturation parameter by providing drops whose volume may be continuously varied (within some range) according to the command given to the dispenser.

One possible motion pattern for three-dimensional printing is a raster pattern. In raster printing, the printhead moves in straight lines along what is referred to as the fast axis. After completion of each pass in the fast axis, the position of the fast axis may be incremented by a specified distance along the slow axis, and another pass is performed along the fast axis.

There is also another, more general possible motion pattern that could be used in three-dimensional printing, which is vector printing. In vector printing, the printhead can move simultaneously in both of the principal (orthogonal) horizontal axes and so can trace curved paths. In such printing, the overall pattern or path of the printing in the part can be curved. It would further be possible to use vector printing in some portion(s) of a matrix and raster printing in other portion(s) of the same matrix.

It is possible to create a sequenced structure having a first region (the innermost or core), followed by a transition region, followed by a second region, where each region is truly three-dimensional. The structure can be convex and axisymmetric, although in general the structure could be any shape. There is no limit as to the number of layers that can be constructed. The powder forming each layer can be deposited by one or more powder depositors which can deposit specific powder compositions in specific places. The individual powder compositions at individual locations within a layer can have individual chemical compositions, such as different polymers, with different contents or concentrations of a porogen such as sodium chloride. In this way, when the porogen is eventually leached out, different porosities remain in the different locations. With the deposition of layers having compositional variation within the layers, different porosities can be produced at different locations within an individual layer of the 3DP process. The individual powder compositions can have either or both of these variations or other variations such as differences in powder particle sizes.

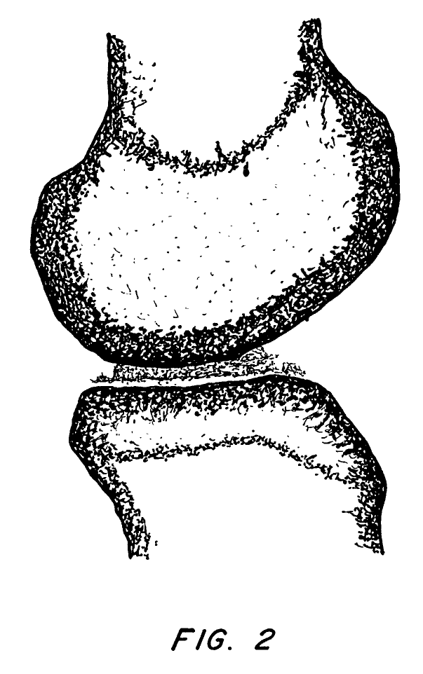

See, for example, FIG. 2, which demonstrates the very complex nature of bone, and how the bone 10 and articular cartilaginous structures 12 are overlaid on each other.

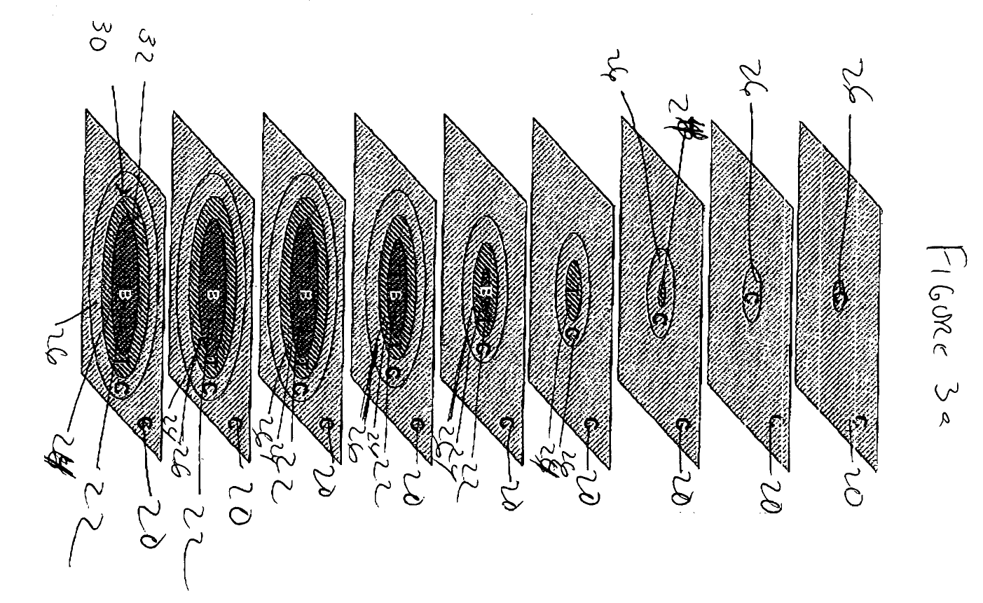

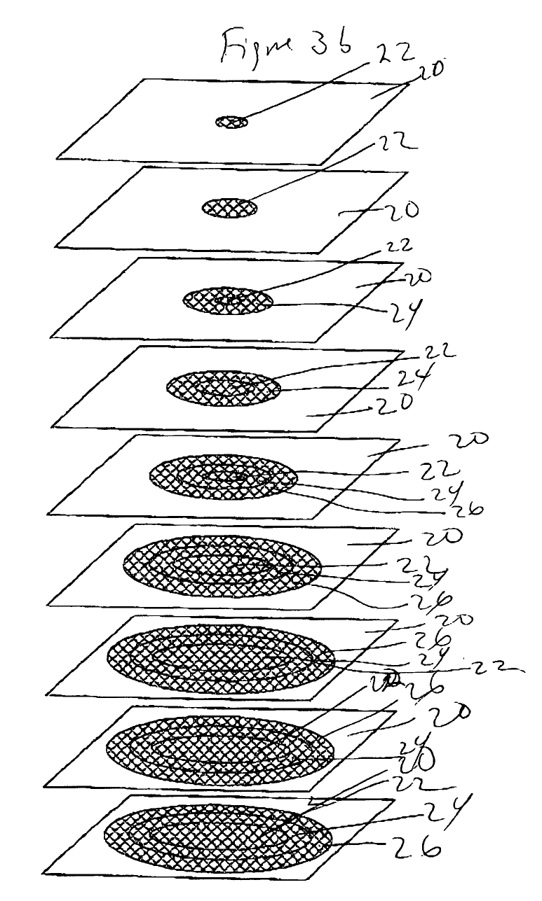

FIGS. 3a and 3b further show how an article which is truly three-dimensional and including complex structure, with convex and concave surfaces (although only convex surfaces are shown), as well as very defined regions, can be made by 3DP. The structure illustrated by these various horizontal sections is a sort of a paraboloid of revolution having an outer curved region which follows the outside shape and is a thin region, followed on the inside by a transition region which follows the shape of the outer region and is a thin region, followed by the interior, which occupies the entire remaining interior of the object and is itself approximately a paraboloid of revolution. Of course, this is only a simple shape for ease of illustration; any general shape, not necessarily with this much symmetry, could be used. For example the method could be used to manufacture at least a portion of a long bone of the human body (humerus, ulna, radius, tibia, fibula, femur, etc.), which would have less symmetry and might even have saddle regions at the ends. FIGS. 3a and 3b illustrate how it is possible to create a sequenced structure 20 having a first region (darkest shading, 22), followed by a transition region (medium shading, 24), followed by a second region (lightest shading, 26), wherein each region is truly three-dimensional.

A vertical section would show that the outermost region, which might be the cartilage region, occupies a curved shape which roughly follows the external contour of the article and is fairly thin compared to its dimensions along the surface of the article. In the article shown here, which approximates a paraboloid of revolution, the surface of the article and also the interior boundary of the outermost region have curvature simultaneously in two mutually orthogonal directions. Such a complicated surface requires that the individual powder layers be able to be deposited with completely arbitrary patterns of composition, as opposed to simple one-dimensional stripes of differing composition. Interior of that outermost region is a middle region. Both the outer boundary and the inner boundary of this middle region are also curved, and specifically are simultaneously curved in two mutually orthogonal directions.

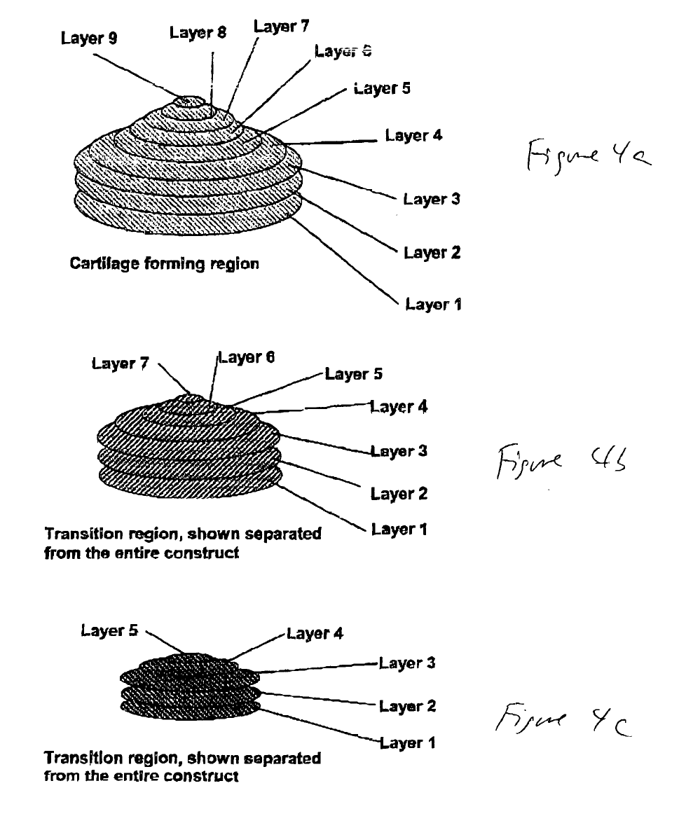

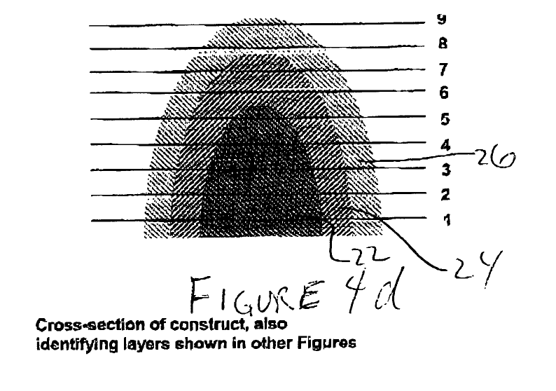

As depicted in FIGS. 4a-c, the structure which is assembled in this manner is convex and axisymmetric, although in general the structure could have any shape. For convenience of illustration, the structure is shown as being exploded into layers; it should be understood that the number of layers illustrated is only for sake of illustration, and in general any number of layers could be used. The layers or sections illustrated could correspond to deposited layers of powder but do not have to so correspond since, for example, more deposited powder layers might be involved in the manufacture than can be conveniently illustrated. For convenience of illustration, three regions and powder compositions (bone, transition region, and cartilage) are illustrated, but other numbers could also be used. Although the regions are discussed in terms of bone and cartilage, it should be understood that in general the device could comprise a region suited for growing any first kind of tissue and a region suited for growing any second kind of tissue and one or more transition regions between them. The shading shows the composition of the powder that would be used on individual layers of the 3DP manufacturing process. The powder forming that layer can be deposited by one or more powder depositors which can deposit specific powder compositions in specific places. The individual powder compositions at individual locations within a layer can have individual chemical compositions such as individual polymers, with different polymers providing different resorption rates or other characteristics. The individual powder compositions can contain individual contents or concentrations of a porogen such as sodium chloride. In this way, when the porogen is eventually leached out, different porosities remain in individual locations. The individual powder compositions can have either or both of these variations or other variations. For example, powder particle size is another possible variation.

FIG. 4a shows the assembled individual regions, separated from each other. A vertical section through the article illustrated in the above layered illustration is shown in FIG. 4d. It can be seen that the outermost region 26, which might be the cartilage region, occupies a curved shape which roughly follows the external contour of the article and is fairly thin compared to its dimensions along the surface of the article. In the article shown here, which approximates a paraboloid of revolution, the surface of the article 26 and also the interior boundary of the outermost region 24 have curvature simultaneously in two mutually orthogonal directions. Such a complicated surface requires that the individual powder layers be able to be deposited with completely arbitrary patterns of composition, as opposed to uniform-composition layers or even simple one-dimensional stripes of differing composition. Interior of that outermost region 26 is shown a middle region 24. Both the outer boundary 30 and the inner boundary 32 of this middle region 24 are also curved, and specifically are simultaneously curved in two mutually orthogonal directions. Interior of the middle region is shown an inner region 22, also having a multiply curved boundary.

B. Materials Used in Manufacture of Devices

1. Materials for Use in Forming the Matrix

The materials used in the manufacture of the devices are biocompatible, bioresorbable over periods of weeks or longer, and generally encourage cell attachment. The term “bioresorbable” is used herein to mean that the material degrades into components which may be resorbed by the body and which may be further biodegradable. Biodegradable materials are capable of being degraded by active biological processes such as enzymatic cleavage. Other desirable properties include (1) solubility in a biologically acceptable solvent that can be removed to generally accepted safe levels, (2) capability of being milled to particles of less than 150 microns, and (3) elasticity and compressive and tensile strength.

One manner in which the process of solid free form fabrication using three-dimensional printing apparatus is used requires that some or all of the structural material of which the final part is to be composed be used in the form of fine particulates or powder. A further characteristic of this method of fabrication is that the minimum final feature dimension of the work product will be dependent on the initial particle size of the powder material used. The process of joining at least two particles by printing a drop of solvent thereon means that the minimum feature size is approximately twice the particle size.

Aggressive solvents tend to nearly dissolve the particles and reprecipitate dense polymer upon drying. The time for drying is primarily determined by the vapor pressure of the solvent. There is a range from one extreme over which the polymer is very soluble, for example, 30 weight percent solubility, which allows the polymer to dissolve very quickly during the time required to print one layer, as compared with lower solubilities. The degree to which the particles are attached depends on the particle size and the solubility of the polymer in the solvent. Fine powder is more quickly dissolved than powder with larger particle size. Furthermore, relatively large particles may not dissolve completely before the solvent binder evaporates.

The device is intended to be manufactured using natural or synthetic structural materials that have inherent ability to encourage cell attachment, such as calcium phosphates, and further provide mechanical integrity to the device in terms of tensile strength and compressibility. The materials must be amenable to milling and sieving to produce specific particle sized powders, spreading of powder, and binding with solvent. Another consideration is the ability to remove free powder from the device post-fabrication.

Materials to be used in the powder bed, if not naturally or otherwise available as substantially uniform particles must be processed to achieve such. Synthetic polymer products used are subjected to cryogenic milling using, for example, an ultra-centrifugal mill (Model ZM100; Glen Mills, Clifton, N.J.) with liquid nitrogen. Analytical milling using such mills as the Model A20, Janke and Kunkel GmbH, Germany, is another preferred technique. Once milled the powders are vacuum dried.

Sieving of the milled material is performed to produce uniformly sized particles of a minimum and maximum size. The maximum particle size will therefore also be a function of the screen used. Screens of about 30 micron mesh are common and other screens of larger mesh may also be employed with satisfactory results. Screens may be stacked on a vibrating sifter-shaker (Model AS200, Retsch, Haan, Germany).

Synthetic polymers which have been found to be particularly useful include: poly(alpha)esters, such as: poly(lactic acid) (PLA) and poly(DL-lactic-co-glycolic acid) (PLGA). Other suitable materials include: poly(ε-caprolactone) (PCL), polyanhydrides, polyarylates, and polyphosphazenes. Natural polymers which are suitable include: polysaccharides such as celluloses, dextrans, chondroitin sulfate, glycosaminoglycans, heparin, or esters thereof; proteins such as chitin, chitosan, and hyaluronic acid and natural or synthetic proteins or proteinoids; elastin, collagen, agarose, calcium alginate, fibronectin, fibrin, laminin, gelatin, albumin, casein, silk protein, proteoglycans, Prolastin, Pronectin, or BetaSilk. Mixtures of any combination of polymers may also be used. Others which are suitable include: poly(hydroxy alkanoates), polydioxanone, polyamino acids, poly(gamma-glutamic acid), poly(vinyl acetates), poly(vinyl alcohols), poly(ethylene-imines), poly(orthoesters), polypohosphoesters, poly(tyrosine-carbonates), poly(ethylene glycols), poly(trimethlene carbonate), polyiminocarbonates, poly(oxyethylene-polyoxypropylene), poly(alpha-hydroxy-carboxylic acid/polyoxyalkylene), polyacetals, poly(propylene fumarates), and carboxymethylcellulose.

Advantages of using PLA/PLGA polymers include clinical experience and acceptance and ease of processing. A disadvantage is the production of acidic degradation products during degradation. However, provision for removal of acidic degradation products, along with other device generated or naturally generated toxins inherently produced during tissue healing or regeneration can be handled by the device design, or by inclusion of buffering agents. PLGA 75:25 degrades rapidly in the body but not as quickly as D,L-PLGA 50:50. PLGA 75:25 degrades in 4 to 5 months whereas D,L-PLGA does so within 1-2 months. On the other hand, other polymers with more slowly degrading properties may be blended with PLGA to produce a device capable of maintaining some physical properties for longer periods of time.

Biologically active materials may also be used to form all or part of the matrix. Osteoconductive materials include: ceramics such as hydroxyapatite (HA), tricalcium phosphate (TCP), calcium phosphate, calcium sulfate, alumina, bioactive glasses and glass-ceramics, animal derived structural proteins such as bovine collagen, and demineralized bone matrix processed from human cadaver bone. Some materials of this nature are commercially available: ProOsteon 500 (Interpore International), BoneSource (Orthofix) and OSTEOSET (Wright Medical Technology), Grafton Gel, Flex, and Putty (Osteotech), and Collagraft (Zimmer).

Hyaluronic acid esters of benzyl or ethyl alcohol have suitable mechanical and degradation properties for use as either cartilage or blood vessel scaffolds and release few degradation products. Hyaluronic acid is present in high concentrations in developing tissues and may confer some potential benefits biologically. Hyaluronate ester powder generation should be possible by the techniques of cryogenic milling or coacervation. Polyethylene oxide (PEO) is available in a wide range of molecular weights and may be used as a blending agent to modify the degradation properties of the polyesters and hyaluronic acid esters.

Inorganic particles such as sodium chloride or tricalcium phosphate may be mixed with the polymer particles in the powder bed. The printing solution used may be a solvent for the polymer or contain a binder and may contain one or more dissolved additional polymers or other substances desired to be incorporated into the component. Preferred solvents are: water, chloroform, acetone, and ethanol.

The binder can be a solvent for the polymer and/or bioactive agent or can be an adhesive which binds the polymer particles. Solvents for most of the bioerodible polymers are known, for example, chloroform or other organic solvents. Organic and aqueous solvents for the protein and polysaccharide polymers are also known, although an aqueous solution is preferred if required to avoid denaturation of the protein. In some cases, however, binding is best achieved by denaturation of the protein. The binder can be the same material as is used in conventional powder processing methods or may be designed to ultimately yield the same binder through chemical or physical changes that take place in the powder bed after printing, for example, as a result of heating, photopolymerization, chemical cross-linking, or catalysis.

It is further possible for some of the powder particles to be a polymer such as PLGA, PLA, polycaprolactone, PMMA, etc., as described elsewhere. The powder particles may be particles of the described substances coated or coacervated with another substance as described below. DBM is not nearly as rigid as natural bone, while most of the ceramic substances are fairly rigid.

The powder from which the matrix is made may comprise any number of the above substances in any combination. Various combinations may be selected to provide desired overall properties as far as stiffness, resorption rate, etc. Different regions of the matrix can have different powder composition. The powder particles may be of aspect ratio reasonably close to spherical or cubical, or, alternatively, at least some fraction of the particles may be of more elongated geometry such as fibrous. The term particle is used herein to refer to all of these shapes.

A binder liquid may cause binding of particles simply by being a solvent for at least some of the particles, so that at least some of the particles dissolve upon application of the solvent and then resolidify upon evaporation of the solvent, as described elsewhere herein. Alternatively, a binder liquid may include a binder substance that is capable of binding the powder particles to each other and to other solid regions when the volatile part of the binder liquid has evaporated. In the matrix, bone augmentation or tissue scaffold matrix, the particles may be bound to each other by at least one binding substance. The binding substance(s) may be collagen or collagen derivatives. Other suitable substances include polymers, which may be either resorbable or nonresorbable. Suitable biocompatible binders include biological adhesives such as fibrin glue, fibrinogen, thrombin, mussel adhesive protein, silk, elastin, collagen, casein, gelatin, albumin, keratin, chitin or chitosan; cyanoacrylates; epoxy-based compounds; dental resin sealants; bioactive glass ceramics (such as apatite-wollastonite), dental resin cements; glass ionomer cements (such as lonocap.RTM. and Inocem.RTM. available from lonos Medizinische Produkte GmbH, Greisberg, Germany); gelatin-resorcinol-formaldehyde glues; collagen-based glues; cellulosics such as ethyl cellulose; bioabsorbable polymers such as starches, polylactic acid, polyglycolic acid, polylactic-co-glycolic acid, polydioxanone, polycaprolactone, polycarbonates, polyorthoesters, polyamino acids, polyanhydrides, polyhydroxybutyrate, polyhyroxyvalyrate, poly (propylene glycol-co-fumaric acid), tyrosine-based polycarbonates, pharmaceutical tablet binders (such as Eudragit.RTM. binders available from Huls America, Inc.), polyvinylpyrrolidone, cellulose, ethyl cellulose, micro-crystalline cellulose and blends thereof; starch ethylenevinyl alcohols, polycyanoacrylates; polyphosphazenes; nonbioabsorbable polymers such as polyacrylate, polymethyl methacrylate, polytetrafluoroethylene, polyurethane and polyamide; etc. Examples of resorbable polymers are starches, polylactic acid, polyglycolic acid, polylactic-co-glycolic acid, polydioxanone, polycaprolactone, polycarbonates, polyorthoesters, polyamino acids, polyanhydrides, polyhydroxybutyrate, polyhyroxyvalyrate, poly (propylene glycol-co-fumaric acid), tyrosine-based polycarbonates, pharmaceutical tablet binders, polyvinylpyrollidone, cellulose, ethyl cellulose, micro-crystalline cellulose, and blends thereof. Examples of nonresorbable polymers are polyacrylate, polymethyl methacrylate, polytetrafluoroethylene, polyurethane, and polyamide. Binder substances may vary in amount or composition from one place to another in the matrix.

In an article containing particles which are not dissolved during the printing process and which are not sintered to each other, the particles are not physically merged with each other as they are in a partially sintered article, but rather may be attached to each other by binder substance. The binder substance may remain in the finished article. Insoluble particles in the powder may become attached to each other by the resolidification of soluble particles of the powder bed which dissolve in the binder liquid after the binder liquid has been dispensed.

In general, it is possible for any component of the matrix to have different composition from one place to another within the matrix, and for more than one composition of any category of substance to be used. The powder composition can vary. The binder substance can vary in composition or concentration from place to place within the matrix. The composition or concentration of strengthening substance, bioactive substance, soluble substance or other substance to vary from place to place within the matrix.

The matrix may have an overall shape that includes geometric complexity. For example, it may include undercuts, recesses, interior voids, etc., as long as the undercuts, recesses, interior voids, etc., have access to the space outside the matrix. The matrix may be shaped appropriately so as to replace particular bones or segments of bones or spaces between bones or voids within bones. The matrix may be dimensioned and shaped uniquely for a particular patient. The matrix can also be modified after completion of the manufacturing steps that give the matrix its shape, such as by a surgeon during an operation. Such modification can be performed by filing, drilling, grinding, or in general any cutting operation or material removal technique.

Three-dimensional printing can also achieve variation of local composition of the powder or solid material within a matrix. One way is the deposition of “stripes” of powder during roller spreading of powder, as has allowed been described. Another is to physically deposit powder particles of specified composition in specified places within a powder layer. Variation of powder composition can be achieved by depositing different compositions of powders in different places in a layer. Varying the powder composition in a matrix provides advantages in terms of biological considerations, such as having both resorbable regions and nonresorbable regions, together with other features.

In one embodiment, layers of powder particles are deposited by dispensing suspension. The various suspensions used in the method may comprise powder particles and a carrier liquid and additives to the carrier liquid. The powder particles in at least one suspension may comprise hydroxyapatite, tricalcium phosphate or other resorbable calcium-phosphorus compounds, polymer particles, and particles of a porogen. A porogen is a material which makes up at least some of the powder particles during three dimensional printing and which, after completion of three dimensional printing, can be leached from the printed article by a suitable solvent, leaving pores in the places formerly occupied by powder particles. Porogens may be soluble in water, so that water is a suitable leaching solvent. A common porogen is sodium chloride. Other suitable porogens are other salts, and various forms of sugar. Porogens are useful for creating three dimensional printed articles which have porosities greater than the porosity typically achievable by three dimensional printing using only non-leachable solid particles in the powder. If the porogen is water-soluble, then the carrier liquid for forming the slurry or suspension may be free or substantially free of water to avoid dissolving the porogen. In general the porogen and the carrier liquid are selected so that the porogen is substantially insoluble or not very soluble in the carrier liquid of the slurry or suspension. A suitable non-aqueous carrier liquid is ethanol or simple alcohols. As is known in regard to suspensions, the powder particles in the suspension may be selected so as to be suitably small so as to have a high likelihood of remaining in suspension. Suitable additives to the carrier liquid, such as steric hindrants or suspending agents or surfactants, may be included to help keep the particles in suspension, such as by preventing them from agglomerating.

The suspension may be delivered to the dispenser or nozzle by a fluid supply system that may include agitation or continuous circulation to help maintain the particles in suspension. Two or more different suspensions each having respective powder compositions may be provided, with each suspension able to be dispensed in appropriate places on a layer. For similarity of dispensing of the respective suspensions, the various fluid parameters which characterize each suspension may be chosen or formulated to be approximately equal to each other, such as viscosity of carrier liquid, additive formulation, particle size, solids content, etc., although this is not absolutely necessary. Typical additives may be added to the carrier liquid to promote suspension. A typical powder particle size for creation of a stable suspension is 40 microns or smaller, dependent on parameters such as density of the particle and composition of the liquid.

Percolation means such as a porous substrate underlying the build bed may be used to promote the drainage of the carrier liquid, as is known in the art. Application of external heat may be used to accelerate the evaporation of the suspension carrier liquid after deposition of a layer has been completed. When the powder in the most recently deposited layer is sufficiently dry, one or more binder liquids, each of which may comprise one or more binder substances, may be dispensed onto that layer in selected places, as is usually done in 3DP, to bind powder particles to each other and to other bound regions. Alternatively, the binder liquid may itself be a solvent for one or more of the substances in the powder. The whole sequence may then be repeated as many times as needed. Possible subsequent processing steps are described elsewhere herein.

The carrier liquid of the suspension, and the binder substance or substances used for the 3DP process (if binding is achieved by a binder substance as opposed to dissolution/resolidification), may be chosen so that the binder substance or substances are not excessively soluble in the slurry carrier liquid. This assures that deposition of suspension for subsequent layers may be performed without appreciably affecting the binding of already-printed layers. For example, the binder substance may be polyacrylic acid and the suspension carrier liquid may be isopropanol or water. Polyacrylic acid is somewhat soluble in isopropanol and water, but not excessively soluble.

A deposited powder layer may be described in terms of its compositional uniformity (comparing the composition of the powder from one place to another) and its geometric uniformity (whether its thickness is essentially constant everywhere). For manufacturing simple articles for industrial products, slurry-deposited layers are typically compositionally uniform because all suspension is delivered from the same source, and effort is made to achieve geometric uniformity as much as possible.

It may be desirable to achieve geometric uniformity of the deposited layer even though the goal is to achieve compositional non-uniformity of the deposited layer. In this regard, it may be desirable that every point on the build bed receives as closely as possible the same amount of deposited suspension as any other point. Depositing a layer by dispensing suspension from a nozzle which is moving relative to the build bed involves typically creating, at the point of impact or deposition, a very slight mound or accumulation of slushy material adjacent to a region which has not yet received a deposit of new material. From at least some directions and for some period of time, the mound may be unsupported. It can be expected that at any impact point the newly-deposited slight mound may have a tendency to migrate or spread, especially in whatever direction and during whatever time period it is not supported by adjacent deposited material of similar height.

A consideration for minimizing migration or spreading of deposited suspension may be to minimize the number of directions from which a mound of deposited slurry is unsupported and the duration of time for which it is unsupported. In this respect, continuous or uninterrupted deposition with constant-velocity relative motion may in general do a better job of minimizing the opportunity for spreading than would a more interrupted type of deposition, and hence would promote the creation of a deposited layer which is as geometrically uniform as possible. Continuous deposition means that to the greatest extent possible there is no interruption in the sense of an impact point being followed in the direction of dispensing motion by a non-impact point. There are several possible ways of creating a location-specific composition of the powder in a layer through appropriate deposition of slurry or suspension (the terms slurry and suspension being used interchangeably herein). In one of these ways, suspension of varying composition may be dispensed from a continuously flowing nozzle. Also, there are at least two ways in which suspension may be dispensed from multiple nozzles in an on-demand manner, with each nozzle being dedicated to a particular composition of suspension.

In conventional slurry deposition, in which a continuously flowing jet is moved in a motion pattern such as a back-and-forth raster pattern, the continuous nature of the rastering means that at least along the fast direction of travel the deposition occurs as continuously as possible. In the present invention, it is also possible that a jet be essentially continuously flowing, and yet the composition of the delivered suspension in the jet can vary with time and hence vary with place of deposition. It can be envisioned that the stream of liquid passing through the nozzle may comprise a bolus of suspension of one composition preceded and followed by suspension of another composition(s). Differences in the composition of suspensions deposited at various locations in the deposited powder layer could be differences in the fraction of porogen relative to other non-leaching solid particles in the suspension. Alternatively, or in addition, there could be differences among different suspensions as far as the composition of the non-leaching solid particles in suspension. Differences in the composition of suspension directed to various locations in the deposited powder layer could be differences in the fraction of porogen relative to other non-leaching solid particles in the suspension. Alternatively, in addition or instead, there could be differences among different suspensions in the composition of the non-leaching solid particles in suspension. Switching between or among dispensed suspension compositions could be performed at any arbitrary time during actual dispensing of suspension over the build bed, which would provide complete opportunity for detailed variation of material composition. Adjustments may be made based at least in part on spatial information as to where the printhead is at a given time, such as from an encoder mounted on the fast axis of the motion control system. There could also be a binder liquid dispenser that may be mounted on part of the same printhead as the suspension dispenser.

Another method of location-dependent suspension deposition involves dispensing of suspension from more than one discrete nozzle or dispenser. This simplifies the fluid supply system in the sense that each individual dispenser or nozzle can be dedicated to a particular suspension composition, and the choice of which suspension composition is deposited at a particular location can be made by the choice of which dispenser is used to deposit the suspension at a particular location. It is possible that two different dispensers may both aim their dispensed suspension at a common impact location on the plane of the build bed.

Appropriate tilting and positioning of the respective nozzles or entire dispensers or both may be used. Controls may be used to ensure that exactly one of the dispensers dispenses at any given point on the build bed, or perhaps more practically speaking, at any given spatial increment into which the build bed may be discretized by the motion control and 3DP system. When changeover of dispensing from one dispenser to the other dispenser is desired to occur, in order to achieve a compositional change, one dispenser stops dispensing and the other dispenser begins dispensing. However, there would be essentially no shift in the impact point of the dispensed suspension, because both dispensers would have the same impact point on the plane of the build bed, and so there would be no disruption in the apparent motion of the impact point on the build bed.

As described elsewhere herein, the dispensers may be a drop-on-demand dispenser such as a piezoelectric drop-on-demand dispenser or may be a microvalve (The Lee Company, Westbrook, Conn.) based dispenser operating in either drop-on-demand or line-segment mode. It is believed that co-aiming will provide continuousness of deposition approaching that of a continuous-flow jet in the same motion pattern, while providing fully detailed control of composition of the deposited layer.

It may not always be possible or desirable to aim two different dispensers at a common location on the plane of the build bed. In this configuration, wherever there is a change of composition of dispensed suspension, there may also be a change in the impact point on the build bed and hence there may be an interruption in the deposition onto the build bed in the sense that where a changeover occurs, the physically next deposition along the direction of motion of the printhead in the fast axis may not follow immediately in time, or may even have already occurred.

If it is necessary to have separate impact points for each individual dispenser, it may be advantageous to have the impact points all be along a single line of deposition along the fast axis direction of motion of the printhead. In this way, all points on a given line will at least receive their deposition of slurry during one pass of the printhead, so that the time interval between receipt of slurry will not be as long as it would be if different passes of the printhead were involved on the same line. This may somewhat minimize any opportunity for unsupported mounds of slurry to spread before becoming more fully supported and should provide the best results achievable within this example.

If migration or spreading of dispensed suspension is not a problem in a particular application, it may be possible to dispense the respective suspensions in a manner in which the dispensings are more independent of each other in time. In this case the various dispensers might not have to be co-located along a line parallel to the fast axis. This may allow more design flexibility regarding the printhead or programming of motion and dispensing commands. Dispensing of suspension may be performed using in general any suitable type of dispenser or printhead that is appropriate to the particular example just given. Dispensing of suspension may be performed with a piezoelectric drop-on-demand printhead or by a microvalve (The Lee Corporation, Westbrook, Conn.) or by a continuous jet with deflection printhead.

Such a dispenser may be designed to have a relatively straight-through flow path having smoothly-varying cross-section, such as may be achieved with a cylindrical-squeeze piezoelectric element, so as to provide as little opportunity as possible for suspended particles to accumulate in isolated places such as corners which might be out of the main path of fluid flow. One mode of microvalve dispensing is to dispense by a succession of brief discrete valve openings, which can be considered drop-on-demand operation. A succession of brief discrete valve openings provides a succession of individual drops if fluid conditions are appropriate, or in some cases provides a succession of fluid packets that may be connected by narrower fluid regions or other fluid geometry. Another possible mode of dispensing with microvalve dispensers, called line-segment printing, is a mode in which a valve opens and remains essentially fully open for as long as needed. In this case the dispensed fluid structure may resemble a steady jet.

Any of these dispensing technologies can be used either with multiple commonly aimed nozzles or with multiple separately aimed nozzles. For the technique involving variation of composition through a given nozzle, microvalves may be used. It has been described that the powder suspended in the first suspension and the powder suspended in the second suspension are in some way of differing composition. It should be understood that each of those suspension powder compositions may individually be somewhat complicated. For example, the powder particles in an individual suspension do not have to all be identical to each other or even be a pure substance. For example, the powder particles in an individual suspension composition may be a mixture of powder particles of more than one substance. It is further possible that an individual powder particle may contain within itself more than one substance. For example, substances of interest in bone applications are the closely related substances hydroxyapatite and tricalcium phosphate, which can transform from one to the other under appropriate conditions of temperature and chemical environment. The same applies to the second suspension composition. The overall composition of the powder of the first suspension is in some way different from the composition of the powder of the second or additional suspension, and the respective suspensions can each be deposited in predetermined locations during the formation of a powder layer for use in 3DP. One or more of the suspensions can includea porogen.

After the deposition of a layer by suspension deposition, carrier fluid may be allowed to percolate downward into the build bed, possibly with the help of a porous substrate underlying the build bed. A drying process with application of heat may be used, if desired, to accelerate evaporation of carrier fluid that does not percolate downward. When a layer of suspension-deposited powder is sufficiently dry, binder liquid may be dispensed onto the layer of powder in places selected so as to form the desired matrix. The binder liquid may be a solvent of at least some of the powder particles or may include one or more binder substances. The steps may then be repeated as needed. The pattern of composition of powder in any particular layer may differ from the pattern in other layers. When an entire matrix has been manufactured and dried, the unbound powder may be removed from it as is known in the art.

There may be still further processing steps such as filling the pores either fully or partially with an interpenetrating substance. The joined powder particles may form a network, and the spaces not occupied by the joined powder particles may form another network that interlocks with the network formed by the joined powder particles. The interpenetrating material may either fully or partially fill that second network. The interpenetrating material may be a polymer, which may be either nonresorbable or resorbable. An example of a nonresorbable polymer is polymethylmethacrylate. Examples of resorbable polymers are poly lactic acid and poly lactic co-glycolic acid. It is also possible, either instead of or subsequent to filling with an interpenetrating material such as a polymer, to fill open spaces with bioactive materials such as cells, cell fragments, cellular material, proteins, growth factors, hormones, Active Pharmaceutical Ingredients, peptides and other biological or inert materials. It may be of interest to fully or partially infuse the matrix with a polymer or a bioactive substance or both.

It has been described that the powder suspended in the first suspension and the powder suspended in the second suspension (or even more suspensions if more are used) are in some way of differing composition. It should be understood that each of those suspension powder compositions might individually be somewhat complicated. For example, the powder particles in an individual suspension do not have to all be identical to each other or even be a pure substance. For example, the powder particles in an individual suspension composition may be a mixture of powder particles of more than one substance. It is further possible that an individual powder particle may contain within itself more than one substance. For example, substances of interest in bone applications are the closely related substances hydroxyapatite and tricalcium phosphate, which can transform from one to the other under appropriate conditions of temperature and chemical environment.

The dispensed suspension as it travels from the nozzle(s) to the build bed may take the form of discrete drops, a continuous jet, an interrupted jet also known as line-segment printing, a series of fluid packets connected by narrower fluid regions, drops with satellite drops, or in general any fluid configuration. Whatever the type of dispenser, dispensing may be performed such that essentially all places on the build bed receive approximately the same amount of dispensed suspension (per unit area) as any other place on the build bed, regardless of which dispenser or suspension source the locally dispensed suspension came from.

When suspension is dispensed by a dispenser moving in a raster pattern, the final surface of the deposited layer after percolation and drying can exhibit a scalloped appearance corresponding to the raster pattern in which slurry was deposited. It is also known that this “scalloping” of the surface can be somewhat reduced by staggering the raster pattern in alternate layers, i.e., depositing lines for the next layer in the valleys of the previous layer. The technique of staggering can be used. Because in the present invention the selection of suspension composition must be coordinated with spatial location of the nozzle, implementing staggering would require an adjustment in the programmed pattern for deposition of individual suspension compositions, to account for the spatial offset in some layers relative to other layers. For example, the pattern of which slurry composition is dispensed where, during given passes, may change as a result of the shifting such as shifting by one-half of the line-to-line spacing of a raster. This can be taken into account in the controls and programming of the 3DP system.

Demineralized Bone Matrix (DBM) is osteoinductive because of its content of organic material, which is more favorable to the ingrowth of natural bone than is the case for ceramic materials, which are merely osteoconductive. DBM could include superficially demineralized, partially demineralized, or fully demineralized bone particles, all of which are included in the term demineralized bone matrix. The particles may all be demineralized bone matrix. Alternatively, some of the particles may be demineralized bone matrix and other particles may be other forms of bone such as nondemineralized (ordinary) bone. The demineralized bone particles and, optionally, nondemineralized bone particles may be obtained from cortical, cancellous, or cortico-cancellous bone of autogenous, allogenic, or xenogenic origin, including porcine or bovine bone. DBM cannot be exposed to temperatures anywhere near as high as ceramics can, or it will decompose. It is further possible that in addition to particles of demineralized bone and possibly ordinary bone, still other substances could be included in the powder particles that are bound together to form the matrix. Examples of such other substances include hydroxyapatite, tricalcium phosphate and other calcium phosphates and calcium-phosphorus compounds, hydroxyapatite calcium salts, inorganic bone, dental tooth enamel, aragonite, calcite, nacre, graphite, pyrolytic carbon, Bioglass.RTM., bioceramic, and mixtures thereof. Hydroxyapatite is generally considered to be nonresorbable by the human body, while tricalcium phosphate and other calcium-phosphorous compounds are resorbable. As discussed elsewhere, the slurry or suspension dispensed to deposit a powder layer can also include particles of one or more porogen, in concentrations which differ from place to place within a deposited layer.

Hydroxyapatite and tricalcium phosphate both occur in natural bone. Hydroxyapatite is generally considered to be nonresorbable by the human body. Tricalcium phosphate is resorbable by the human body over a time period of months. Other calcium-phosphorus compounds are also resorbable.

Possible forms of matrix include replacements for the entirety or portions of essentially any bone in the human body, or augmentations or reconstructions thereof, or bones in animals, including but not limited to craniofacial, alveolar ridge, mandible, parts for spinal fusion, legs, arms, hands, feet, joints, etc.

Resorbable polymers that are members of the polyester family, such as poly lactic acid (PLA) and poly lactic co-glycolic acid (PLGA). Other members of the polyester family are homopolymers (lactide), copolymers (glycolide), and terpolymers (caprolactone), and L-PLA, poly (D,L-lactide-co-glycolide) (D,L-PLA) and PCL (poly(epsilon-caprolactone)), poly(glycolic acid) (PGA), poly(L-lactic acid) (PLLA) and their copolymer, poly(DL-lactic-co-glycolic acid) (PLGA). The biocompatibility and sterilizability of these polymers have been well documented. In addition, their degradation rates can be tailored to match the rate of new tissue formation. The degradation rate of the amorphous copolymer can be adjusted by altering the ratio of lactide monomer to glycolide monomer in the polymer composition.

It is known that when PLGA and similar substances erode, they erode in a bulk fashion. It is possible for significant quantities of such substances to disappear or collapse around the same time, which is not ideal for bone in-growth. For bone in-growth it is desirable for bone to in-grow at essentially the same rate at which implanted material disappears. Thus, any sudden or rapid disappearance of implanted material is undesirable, and gradual disappearance is preferred. However, polyesters are not the only possible family of materials. There are other known materials that disappear gradually by an erosion diffusion process, which means that the material can only disappear from the outside or surface working its way inward. An example of such a material is polyhydroxyalkanoate (PHA). Polyanhydrides exhibit bulk surface degradation and dissolution.

Comb polymers may be used as the polymeric material making up at least some of the powder particles. Different comb polymers could be deposited in different regions of the biomedical matrix. In general, different polymers of any type could be deposited in different regions of the biomedical matrix. They could be deposited in any combination of comb polymers or ordinary polymers and any combination of resorbable or non-resorbable polymers.

2. Incorporation of Auxiliary Materials or Bioactive Agents

Appropriate surface chemistry or biological factors or growth factors positioned on or in the device and releasable in a physiological environment for the purpose of stimulating cell attachment, growth, maturation, and differentiation in the area of the device is readily achievable using the methods described herein. Those bioactive agents that can be directly dissolved in a biocompatible solvent are most preferred. Examples generally include proteins and peptides, polysaccharides, nucleic acids, lipids, and non-protein organic and inorganic compounds. As used herein, “bioactive agents” have biological effects including, but not limited to, growth factors, differentiation factors, steroid hormones, cytokines, lymphokines, antibiotics, and angiogenesis promoting or inhibiting factors.

Bioactive agents also include compounds having principally a structural role, for example, hydroxyapatite crystals in a matrix for bone regeneration. The particles may have a size of greater than or less than the particle size of the polymer particles used to make the matrix.

It is also possible to incorporate materials not exerting a biological effect such as air, radioopaque materials such as barium, or other imaging agents for the purpose of monitoring the device in vivo.

In order to promote cell attachment, cell adhesion factors such as laminin, pronectin, or fibronectin or fragments thereof, e.g. arginine-glycine-aspartate, may be coated onto or attached to the device. The device may also be coated or have incorporated therein cytokines or other releasable cell stimulating factors such as; basic fibroblast growth factor (bFGF), transforming growth factor beta (TGF-beta), nerve growth factor (NGF), insulin-like growth factor-1 (IGF-1), growth hormone (GH), multiplication stimulating activity (MSA), cartilage derived factor (CDF), bone morphogenic proteins (BMPs) or other osteogenic factors, and angiogenesis modulating factors (which may inhibit angiogenesis, such as angiostatin, or enhance angiogenesis, such as vascular growth factor, VGF).

Either exogenously added cells or exogenously added factors including genes may be added to the implant before or after its placement in the body. Such cells may include autograft cells which are derived from the patient's tissue and have (optionally) been expanded in number by culturing ex vivo for a period of time before being reintroduced. Cartilage tissue may be harvested and the cells disaggregated therefrom, and cultured to provide a source of new cartilage cells for seeding the devices. The devices may be seeded with cells ex vivo and placed in the body with live cells attached thereto, seeded at the time of implantation, or cells can be allowed to ingrow following implantation.

An implant can be seeded at the time of implantation or before implantation. A simple way of seeding is to place the implant in a suspension of one or more types of cells. By selection of the pore size, porosity, and composition, one can bias the type of cell that will attach to the implant. This is referred to as “directed cell attachment”. The parameters for cartilage and bone forming cells are known and published in the literature or herein; the parameters for other cell types are readily determined, either from the literature, or simple screening techniques by placing small discs of various compositions and structures into suspensions of the different cell types.

DNA of a gene sequence, or portion thereof, coding for a growth factor or other of the auxiliary factors mentioned above may also be incorporated into the device or added to the device before or after placement in the body. The DNA sequence may be “naked” or present in a vector or otherwise encapsulated or protected. The DNA sequence may also represent an antisense sequence of a gene or portion thereof.

There are two possible methods for incorporation of bioactive agent into the device: (1) as a dispersion within a polymeric matrix and as (2) discrete units within a discrete polymeric matrix. In the first method, the bioactive agent preferably is applied in the polymer particle binder; in the second method, the bioactive agent is applied in a non-solvent for the polymer particles. The selection of the solvent for the bioactive agent depends on the desired mode of release and the compatibility of the bioactive agent in the solvent. The solvent is selected to either dissolve the matrix or is selected to contain a second polymer that is deposited along with the bioactive agent. In the first case. the printed droplet locally dissolves the polymer powder and begins to evaporate. The bioactive agent is effectively deposited in the polymer powder after evaporation since the dissolved polymer is deposited along with the agent. The latter case, where both the drug and a polymer are dissolved in the printed solution, is useful in when the powder layer is not soluble in the solvent. Binding is achieved by deposition of the binder, in this case the polymer, at the necks between the powder particles so that they are effectively bound together along with the bioactive agent.

Devices may be fabricated with bioactive-rich regions within the device. In this case, multiple printheads are used to deposit active containing solvent in selected regions of the powder bed. The remaining volume of the desired device is bound with pure solvent deposited by a separate printhead. The devices also simply may be coated with the bioactive agent or have the agent placed therein or thereon. The bioactive agent may be covalently or noncovalently attached to the device.

The bioactive agents can be processed into particles using spray drying, atomization, grinding, or other standard methodology. Those materials which can be formed into emulsions, microparticles, liposomes, or other small particles, and which remain stable chemically and retain biological activity in a polymeric matrix, are preferred.

Bioactive substances which can be readily combined with the bone particles include, e.g., collagen, insoluble collagen derivatives, etc., and soluble solids and/or liquids dissolved therein; antivirals, particularly those effective against HIV and hepatitis; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamicin, etc.; biocidal/biostatic sugars such as dextran, glucose, etc.; amino acids; peptides; vitamins; inorganic elements; co-factors for protein synthesis; hormones; endocrine tissue or tissue fragments; synthesizers; enzymes such as collagenase, peptidases, oxidases, etc.; DNA delivered by plasmid or viral vectors; growth factors such as bone morphogenic proteins (BMPs); osteoinductive factor; fibronectin (FN); endothelial cell growth factor (ECGF); cementum attachment extracts (CAE); ketanserin; human growth hormone (HGH); animal growth hormones; epidermal growth factor (EGF); interleukin-1 (IL-1); human alpha thrombin; transforming growth factor (TGF-beta); insulin-like growth factor (IGF-1); platelet derived growth factors (PDGF); fibroblast growth factors (FGF, bFGF, etc.); periodontal ligament chemotactic factor (PDLGF); and somatotropin; immunomodulatory agents, and chemotherapeutic agents. These may vary in amount or composition from one place to another in the matrix, and more than one such substance may be used.

The present invention will be further understood by reference to the following non-limiting examples.

### EXAMPLE 1

Polymeric Components with Channel Architecture

The development of devices designed specifically to encourage cartilage regeneration with respect to materials selection and macroscopic architecture is described in PCT/US99/23732. The materials composition was selected to yield a high porosity and to degrade within several weeks. Two primary polymer combinations involving PLGA and PLA were evaluated for their use in cartilage devices. Two variants of macroscopic staggered channel architectures were developed. The objective of the macroscopic channels was to facilitate cell seeding and proliferation. The desired macroscopic channel size was chosen to be approximately 200 μm to maximize the surface area available for cell seeding without compromising structural integrity or homogeneous tissue formation.

Cartilage Batch A

This batch of cartilage devices, referred to as Batch A, included a 1:1 ratio of D,L-PLGA (50:50) 50,000 MW (Boehringer Ingelheim) with free acidic side chains to L-PLA 27,000 MW (Birmingham Polymers). The polymer particle size was 63-106 microns. PLGA with free acidic side chains was chosen to increase the rate of degradation of the device since previous results with standard PLGA suggested that faster degradation may be desirable. A 90 wt % NaCl and 10% PLA-PLGA mixture was used to obtain high porosity. The pore sizes were expected to be larger than the NaCl particle size, which was 106-150 mm. After leaching on an orbital shaker at 37° C. for 48 hours, these devices shrank 8.3% in diameter and 20% in thickness. The disks were fully leached after 7 hours, according to the silver nitrate assay, with a 90% weight loss (i.e., porosity). No residual chloroform was detected in these disks (n=5).

Batch A contained staggered channels that did not fully go through the thickness of the device. This was to model the cartilage-bone composite device in which the bone region will not contain macroscopic channels. The macroscopic staggered channel architecture was created with layers containing grooves traversing the diameter (or arc) of the disk. The bottom layer contained no macroscopic channels. Grooves were formed by not depositing chloroform on sections 0.675 mm in width within the layer. The grooves were spaced 2.05 mm apart. Sixteen holes were constructed on the top face of the device superposed over the grooves. These holes were formed by printing a layer of grooves, rotating the print bed 90°, and printing another set of grooves without spreading additional powder. This effectively double-printed a significant portion of device matrix with chloroform. Double-printing may also improve mechanical properties of the final device by more completely dissolving the polymer and thus create a stronger bond between the polymer particles. The channel size was observed to be 182±37 μm in the actual devices. The drawback of this architecture design is that the two sets of grooves lie parallel to each other, potentially causing a structural weakness. This was not a critical concern if the devices are to be seeded statically.

The scanning electron micrograph (Evans East, Plainsboro, N.J.) of the cross-section shows evidence of the staggered channel architecture. Protruding walls separated by channels are outlined. Some of the features were lost upon sectioning the device. The SEM of the surface also reveals the porous network, which includes primary pores that were greater than 100 microns and secondary pores less than 10 microns in size.

Cartilage Batch B

A batch of cartilage devices, referred to as Batch B, was fabricated as a stand-alone cartilage replacement product. The devices therefore needed to be of sufficient strength to withstand the fluid flow during culture conditions in a bioreactor. Batch B was similar to Batch A but some improvements were made in the materials composition and the macroscopic architecture to satisfy these performance requirements. To minimize the pressure build up from fluid flow, macroscopic channels running completely through the device were used. In addition, supporting walls were used in the layers containing long grooves, and these grooved layers were offset 90° from each other. The materials and architecture of these devices were the same as those used in the cartilage region of the cartilage-bone composites.

After leaching for 48 hours, the devices shrank 5.3% in diameter and 7% in thickness. After leaching for 7 hours, the devices were fully leached according to the silver nitrate assay. These devices were estimated to be 90% porous based on the weight change from leaching which is in agreement with the design planned. Residual chloroform analysis, which has a lower detection limit of ˜50 ppm, suggests a negligible amount of chloroform was present (n=4).

Differential scanning calorimetry was performed on batches fabricated of devices containing a 1:1 ratio of D,L-PLGA and L-PLA. Since D,L-PLGA is amorphous and L-PLA is crystalline, these devices had both glass transition temperatures and melting temperatures. All batches had a glass transition temperature of 53° C. and melting temperature of 161° C. (n=3) which demonstrates consistent physical properties between fabrication runs.

### EXAMPLE 2

Composite Device for Cartilage and Bone Regeneration.

Devices having structures consisting of an upper cartilage component, a transition zone, and a lower bone component for insertion and anchoring into the underlying bone of osteochondral defects were described in PCT/US99/23732. The materials to be used in the bone portion of the cartilage-bone composite are a slow degrading PLGA, tri-calcium phosphate (CaP), and NaCl. The NaCl was leached out to form micropores in the final device.

Materials and Methods

A trial batch of cartilage-bone composite devices was fabricated with a bone region, a transition region, and a cartilage region with macroscopic channels identical to that of Cartilage Batch A. The overall dimensions of the product were 8 mm×1 cm before drying and salt leaching. The objective of this development batch was to evaluate the lamination and mechanical integrity of the final device.

Cartilage-Bone Composite Design Description

Sixteen staggered channels were incorporated into the microarchitecture of these devices. The channels were a nominal 0.675 mm square and were spaced 2.05 mm. Two layers of channels were separated by three layers of walls, 1.375 mm wide and spaced 2.05 mm. A detachable print plate was used to allow rotation of the powder bed underneath the stencil. Each channel layer included printing on the non-rotated and the rotated powder bed. A manual roller was used to spread powder.

Five different polymer combinations were used in the powder bed to produce cartilage-bone disks. The sequence was as follows: 3 layers of stilts, 22 layers of bone region, 6 layers of transition region, and 10 layers of cartilage region using staggered channels. Double-sided tape was applied and stilts were constructed of three layers 200 μm each. Stilts were printed in a crosshair configuration, with two adjacent lines per leg. The polymer combination for region 1 made up the stilts and the bone portion of the device (layers 1 to 22). A 1-cm cloverleaf stencil was used for the bone and first two transition regions. The bone region was made of one powder composition, each of the 3 transitions regions (2 layers each) were made with different powder compositions, and the cartilage region had a fifth powder composition.

A circular stencil was used for the last transition region and the cartilage region. The osteochondral scaffolds consisted of three distinct regions. The bone region was 4.4 mm high and fabricated with 33.75 wt % L-PLGA(85:15) I.V. 1.45 dL/g (Birmingham Polymers Inc., Birmingham, Ala.) milled to 38-150 microns, 11.25 wt % TCP (Sigma) 38-106 um, and 55 wt % NaCl (Fisher) 125-150 microns. The bone region was shaped as a cloverleaf. The cartilage region was 2 mm tall and fabricated with 5 wt % D,L-PLGA(50:50) I.V. 0.48 dL/g (Boehringer Ingelheim, Germany) and 5 wt % L-PLA I.V. 0.34 dL/g (Birmingham Polymers Inc.), both milled to 63-106 um, and 90 wt % NaCl that was 106-150 microns. Staggered channels that were approximately 250 microns were incorporated into the cartilage region. The transition region (1.2 mm) consisted of three sections: 65 wt %, 75 wt %, and 85 wt % NaCl with 30 wt %, 15 wt %, and 5 wt % L-PLGA(85:15), respectively. The balance of the transition sections was composed of a 1:1 ratio of D,L-PLGA (50:50) and L-PLA.

The powder combination for region 5 made up the cartilage portion of the device, which included 10 layers of channel architecture. Construction of channels required printing on a layer then rotating the plate 90° and then printing again on the same layer (in a specific pattern). The top right corner of the plate was registered to the walls of the piston housing. The 16 channels arranged in a 4×4 array, were nominally 0.675 mm square and were spaced 2.05 mm apart. Two layers of channels were separated by two layers of transition channels. The transition channels were similar to normal channels, but were nominally 0.675 mm wide and 1.90 mm long.

The resulting cartilage-bone composite devices included a unique macroscopic architecture in addition to the gradients of materials. The bottom of the device was approximately 5 mm thick and was fabricated with a cloverleaf stencil for enhanced bone ingrowth. The next six layers included the transition region with the bottom four layers using the cloverleaf stencil. The top two layers of the transition region used the disk stencil to avoid mechanical strength concerns. The top 2 mm of the composite, the cartilage region, was fabricated with macroscopic staggered channel architecture. Minor modifications were made to enhance the structural integrity of the device. For increased support, thin walls were added in the long grooves. The grooves were also rotated 90° with respect to each other.

The fabrication parameters, machine settings, and materials producing the best results for the bone-composite device are shown below.

Printing Parameters: flow rate: 1.2 ml/min; reservoir P: 18 psig; print speed: 125 cm/s; line spacing: 125 μm

Materials: Binder=Solvent: 100% chloroform (Fisher Scientific)

Several different material compositions were incorporated into the composite device structure to form the bone, transition, and cartilage regions. The materials were chosen to minimize the detrimental effects of shrinkage. Variables that were fixed were 90% NaCl content for the cartilage region and leaching temperature (temperature used for cell culture).

Finishing

The large size of the composites (8 mm in height) necessitated leaching for periods much longer than previous disk devices. It was discovered that during exposure to prolonged leaching (>24 hours), the cartilage region delaminated between the cartilage and transition regions when the cartilage region was composed of D,L-PLGA without acidic side-chains. The cause of the delamination was attributed to a significant level of differential shrinkage between these two regions. The adjacent transition region was found to only shrink 3.8% in diameter compared to the 8.3% of the cartilage region. This caused excessive shear stress and eventually resulted in delamination. This level of shrinkage was not encountered before, and changes in either the leaching process or device composition may have contributed to the delamination.

The most favorable candidate for cartilage device fabrication as determined by the shrinkage study was the use of PLGA without acidic side chains and CO₂ drying before leaching. A 1:1 ratio of D,L-PLGA (50:50) 50,000 MW and L-PLA 27,000 MW was used for the cartilage region. The transition region included a gradient of NaCl from 85% to 65%, of 1:1 PLGA:PLA from 10% to 5%, and a gradient of L-PLGA (85:15) 242,000 MW from 5% to 30%, from the cartilage region to the bone region. The bone region was fabricated with 55% NaCl and a 3:1 ratio of PLGA (85:15) to TCP. This was chosen as the presumed optimal composition for osteoconduction and mechanical strength. The composite devices were incubated in 37° C. static PBS solution for a period of one month to verify mechanical integrity. No delamination or other defects were observed.

Performance of the device design. Macroscopic staggered channels in the cartilage portion of the device allow chondrocytes to be seeded in vitro throughout the thickness of the device, not just on one surface. This is important for cartilage formation since chondrocytes cannot migrate easily over distances larger than about 2 mm. Thus, the staggered channel design facilitates chondrocyte seeding directly into the center of the cartilage portion of the device. More homogeneous seeding promotes faster homogeneous cartilage formation. In association, the staggered channels facilitate the transport of nutrients to the cells and removal of cellular by-products and polymer degradation by-products away from the cells during culture in cell growth media. The bone implantable portion of the device does not have staggered channels for two reasons: osteocytes are highly migratory and therefore do not need such a configuration and to impart mechanical strength to this portion of the device. The latter property is an important characteristic enabling the device to withstand the forces of surgical implantation.

In vitro tissue formation by numerous cell types was tested on biodegradable or biostable synthetic scaffolds to engineer dermis, cartilage or smooth muscle for human transplantation. Scaffolds differed by their chemistry, structure (e.g., dimensions, architecture, pore size, or void fraction [VF]) and fabrication (e.g., woven, knitted, felted, braided, solvent cast as sponges, or TheriForm™ processed [i.e., 3-D printed]). Materials included nylon, poly(glycolic acid), poly(ethylene terephthalate), poly(ε-caprolactone), poly-L-lactic acid or poly(D,L-lactide co-glycolide)/poly(L-lactic acid). Human- or animal-derived cells (dermal and arterial fibroblasts, keratinocytes, articular chondrocytes, arterial smooth muscle cells and arterial endothelial cells) were cultured on scaffolds statically or dynamically for up to eight weeks. Analyses were customized per engineered tissue (quantitative MTT and DNA tests for metabolic activity and cell number, respectively; DMMB assay for glycosaminoglycans, Sirius Red assay for collagen, image analyses for pre- and post-culture dimensions, scaffold and tissue mechanics, and qualitative immunostaining and histology).

The data showed that human and animal cell types adhered to, proliferated and readily produced tissue within scaffolds of various chemistries; however, the ingrowth, distribution, orientation, and viability of cells and the gross morphology of constructs were influenced by both cell type and scaffold features (pore size, VF, fiber density, degradation). The depth and uniformity of colonization and amount of extracellular matrix formed by chondrocytes, fibroblasts, smooth muscle cells and endothelial cells corresponded to the pore size in TheriForm scaffolds. Fibroblast orientation in felts and braids followed the random or linear polymer fiber arrangement, respectively. Fibroblasts on nylon meshes formed monolayers or 3-D tissue depending on the particle sieve size. By prescribing scaffold features, one can regulate the cellular destination, orientation and extracellular matrix production on scaffolds in vitro to consistently form viable, confluent tissues for transplantation.

### EXAMPLE 3

Effect of Salt Concentration and Resulting Porosity

Articular cartilage defects have a limited ability to heal. Tissue engineered constructs made by growing cells on highly porous PGA scaffolds have been used to repair osteochondral lesions. The macroscopic architecture of scaffolds used in tissue engineering can have a dramatic affect on the cellular incorporation and matrix deposition. This study was designed to examine the effect of scaffold porosity and pore size on chondrocyte attachment, growth, and formation or deposition of a cartilage specific extracellular matrix.

Materials and Methods:

PLLA scaffolds of varying porosity and pore size were fabricated using the three-dimensional printing process (TheriForm™). The macroporous structure in the scaffolds was created by incorporation of a porogen, NaCl, followed by leaching of NaCl from the scaffolds. The porosity of the scaffolds was controlled by altering the weight ratio of polymer to NaCl particles incorporated into the scaffold. Eight batches of PLLA scaffolds were manufactured. Of the eight batches four were made with a salt fraction of 75% and four were made with a 90% salt fraction, resulting in scaffolds having an approximate porosity of 75% and 90% porosity, respectively. In addition, scaffold pore size was controlled by using NaCl of specified particle sizes in the fabrication process. The NaCl particles used in the scaffold fabrication were seived into sizes <38, 38-63, 63-106, and 106-150 microns to create scaffolds with pore sizes defined by these particle sizes. One batch of scaffolds was made at each pore size range for each of the two porosities. Scaffolds were 10 mm in diameter and 2 mm thick. PGA entangled meshes were used as control scaffolds and have an approximate porosity of 97% and fiber spacing of 90 microns. All scaffolds were seeded on one side with 4e6 primary ovine articular chondrocytes (OAC) from juvenile sheep via a bidirectional syringe method and cultured for 4 weeks in a bioreactor system. Cell-seeded constructs were harvested post-seed for functional cell distribution by MTT and total cell number by DNA analysis. Constructs harvested after 4 weeks of culture were analyzed for MTT staining as well as DNA, sulfated glycosaminoglycan (S-GAG), and collagen content.

Results:

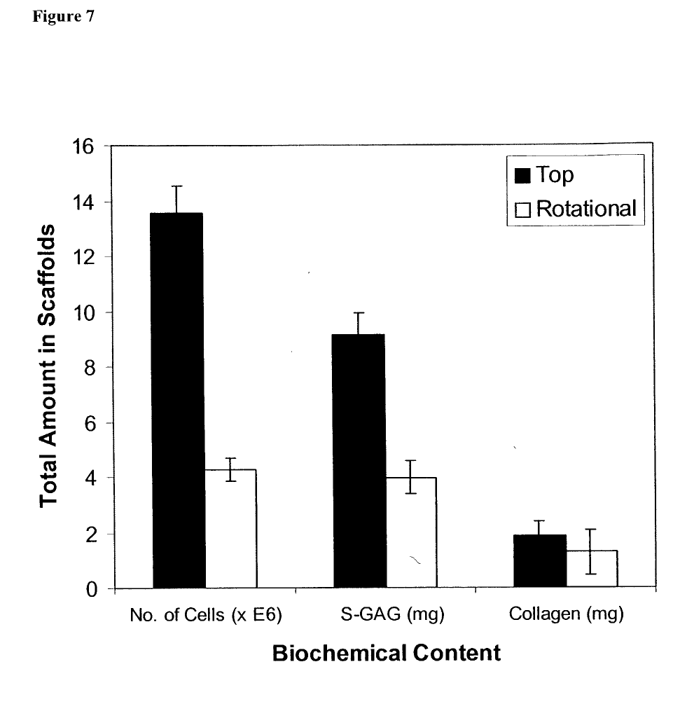

Chondrocytes were found to attach, grow, and deposit hyaline-like matrix in all scaffolds studied. The 90% porous scaffolds supported more uniform cell seeding than the 75% porous scaffolds, for all pore sizes, as demonstrated by MTT stained samples. By four weeks in culture, the cells had proliferated to over 5 fold of their original numbers in the 90% porous scaffolds and to a lesser extent in the 75% porous scaffolds. Greater amounts (p<0.01) of sulfated-GAG and collagen (FIG. 7) were found in the 90% scaffolds compared to the 75% porous scaffolds. Similar amounts of S-GAG and collagen were found in the 90% Theriform™ scaffolds as the PGA control scaffolds (FIG. 7). Examination of histological samples also confirmed that more cartilagenous matrix was produced in the 90% porous scaffolds. Pore size of the scaffolds did not have a significant effect on any of the quantitative measurements (DNA, S-GAG, and collagen) for both porosities. Nevertheless, scaffolds of both porosities allowed for more homogeneous cell seeding and uniformly distributed matrix with increasing pore size.

Tissue engineered constructs may be modified by controlling the scaffold architecture. TheriForm™ scaffolds composed of 90% porous PLLA contained equivalent cartilage matrix levels as compared to PGA scaffolds. In contrast, chondrocytes deposited much less (p<0.05) hyaline-like matrix in the 75% porous TheriForm scaffolds. More uniform cell seeding and deposition of safranin-O stained matrix was found in the scaffolds of greater pore sizes. This study demonstrated that scaffolds of various porosity and pore size can have a dramatic effect on the extent and uniformity of cell seeding and matrix deposition, suggesting that these two parameters can be altered in order to either promote or limit the incorporation of cells or ingrowth of tissue.

### EXAMPLE 4

Preparation of a Cartilage Implant.

Studies were aimed at: 1) the selection of the appropriate polymeric material for the cartilage region, 2) mechanical testing of the bone region including the effect of porosity and polymer/calcium phosphate ratio, 3) prevention of delamination in the transition region, and 4) selection of an appropriate chondrocyte seeding method that results in high matrix deposition in the cartilage region but little in the bone region.

Materials and Methods

Solvent Casting and Testing of Thin Films

To initially screen polymer combinations and molecular weights, thin films were cast. In 7-mL glass scintillation vials, 200 mg of polymer (as received) was dissolved in 2 mL of chloroform. The solutions were mixed and placed on an orbital shaker until the polymer completely dissolved. The solutions were mixed again immediately before being poured into a 6-cm diameter glass Petri dish. The films were allowed to dry covered and undisturbed for 48 hours in a laminar flow hood. After drying, the films were peeled from the bottom of the dishes and statically incubated in phosphate buffered saline (PBS) at 37° C. for three weeks. A sample was taken and qualitatively evaluated once weekly for color (e.g., clear or white), rigidity (e.g., brittle or flexible), structural integrity (e.g., tears, crumbles, or remains intact when collecting a sample), and amount of degradation (e.g., partially or completely degraded).

Powder Preparation

Polymer powders were cryogenically milled in an ultra-centrifugal mill (Model ZM 100; Glen Mills, Clifton, N.J.) with liquid nitrogen. The powders were vacuum-dried and hand-sieved with stainless steel sieves (W. S. Tyler Co., Mentor, Ohio). NaCl was prepared by milling in a large analytical mill (Model A20; Janke and Kunkel GmbH, Germany) at 20,000 rpm and sieved to the specified range within 106-150 μm. Calcium phosphate tribasic (TCP; Sigma, St. Louis, Mo.) was sieved to 38-106 μm as received. The powders were sieved using Retsch screens (Retsch, Haan, Germany) along with zirconia milling media. The stack of screens was placed on a vibrating sifter-shaker (Retsch) and shaken for 15 minutes to separate the powders based on particle size. The powders were mixed on a ball mill (US Stoneware, East Palestine, Ohio).

Scaffold Fabrication Using the TheriForm™ Process

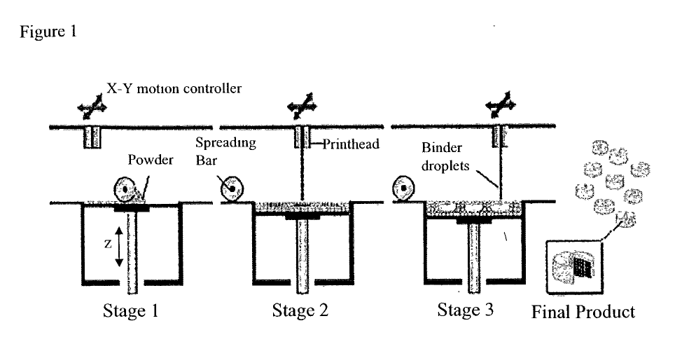

The TheriForm™ process is CAD/CAM driven and selectively binds powder particles with a liquid binder to form solid three-dimensional objects one layer at a time. FIG. 1 is a schematic of a laminated process in which a thin layer of powder is spread and then bound together in desired areas with a liquid binder. External shapes (e.g., cloverleaf) and internal architectural features (e.g., channels) are created via CAD software. During fabrication, a thin layer of powder (polymer/NaCl or polymer/NaCl/TCP) was spread on a piston plate and a printhead rastered above the powder bed and deposited chloroform (Fisher Scientific, Pittsburgh, Pa.) droplets in selective areas to create the scaffold. After one layer was complete, the piston plate was lowered and a new layer of powder was spread, followed by additional deposition of binder (chloroform). The lamination process was iterated until fabrication was complete. The fabrication of these research-grade prototypes was aided by the use of templates for the outer shape (e.g., cloverleaf). The plate of parts was dried overnight at room temperature and the loose powder was removed to reveal the final scaffolds. Residual chloroform was removed with liquid CO₂ and the NaCl was leached to create the micro-pores, as described below.

Solvent Extraction Using Liquid CO₂

Samples were loaded and sealed into the extractor chamber (Marc Sims S.F.E., Berkeley, Calif.). The system was filled with liquid CO₂ and pressurized to 4,000 psi. The system was held for approximately 10 minutes and was vented for 10 minutes at constant pressure. The typical venting rate was 5 standard cubic feet per minute (scfm). The venting-down phase was then initiated. This process was repeated twice per batch.

NaCl Leaching

After removal of residual chloroform, samples were placed into a NALGENE® bottle that contained a minimum of 20 mL of water per sample. The bottle was placed onto an orbital shaker (model 3527, Lab-Line Environ, Melrose Park, Ill.) at 100 rpm and 37° C. or room temperature. The water was replaced every hour. After five hours, the NaCl content in the solution was checked by adding a few drops of 0.1 N silver nitrate (observation of a white precipitate indicated presence of NaCl). If NaCl was detected, leaching was continued until none was detected (approximately 9 hours). Samples were removed, blotted dry, and placed into a vacuum desiccator overnight to complete drying.

Residual Solvent Analysis

Residual chloroform analysis was performed by gas chromatography using a flame ionization detector (GC-FID, Shimadzu GC-14, Shimadzu Instruments, MD). The method was based on the USP Organic Volatile Impurities method <641> and used a Rtx-1301 wide-bore glass column (Restek, 30-m long, 0.53-mm ID, 3.0-μm film thickness) with helium as the carrier gas.

Scanning Electron Microscopy Analysis

Evans East, Plainsboro, N.J. performed the scanning electron microscopy (SEM) analysis of polymer scaffolds. The scaffolds were carefully sectioned along the channels with a razor blade and mounted onto aluminum stubs. Prior to examination, each sample was gold coated. A JEOL 5300 SEM microscope at 20 kV was used to perform image analysis. Polaroid micrographs were taken of both surface and cross-sectional views of each sample.

Mechanical Testing of the Bone Region

The mechanical properties of the bone portion of the osteochondral device were investigated by performing mechanical testing on dog bone-shaped and cylindrical parts made of L-PLGA(85:15), TCP, and NaCl using the TheriForm process. The TCP was used in the 38-150 μm particle size range, and NaCl (Fisher) in the 75-150 μm size was used. Samples of five different compositions were fabricated to study the influence of porosity and inorganic content on tensile and compressive properties. The tensile specimens were twenty 200-micron layers thick, and the compression samples were sixty 200-μm layers. Samples were liquid CO₂-dried to remove residual chloroform, leached (200 mL water per sample) for 15 hours (changing the water every 5 hours) and dried for 48 hours in a vacuum oven (at 1 bar) at room temperature before testing.

Determination of values for elastic modulus, yield strength, tensile strength, percent elongation and compressive strength were obtained from load-displacement curves, briefly described below. Tensile testing specimens were fabricated with dimensions conforming to ASTM standard D 638-96. An Instron Testing machine (model 4201, Instron, Canton, Mass.) was used for both tensile and compression testing. Pneumatic grips (Instron type 2712) were used to hold the specimens in place with an external air pressure of 30 psi. This pressure produced some deformation of the wide section of the sample. To ensure good transfer of load from the grips to the specimen, it was necessary to use a spacer on the far edge of the grips. A strain rate of 0.1 mm/min was applied on five replicates and the load was recorded during the process. Displacement was measured using extensometers (Instron, Cat. no. 2620-826, travel±0.254 mm) with plasticine underneath. The elastic modulus was calculated as the ratio of stress to strain before the material yielded, using the initial cross-sectional area in the calculations. Tensile strength was found as the peak stress before fracture.

Compression testing was carried out according to the ASTM standard D 695-96. This protocol recommended using a cylindrical specimen with a length twice its diameter. Cylindrical samples were fabricated having diameters of 6 mm and lengths of 12 mm for use in this study. Five replicates of each composition were subjected to this test using the same Instron as for the above tensile tests. After removing surface aberrations using fine sandpaper, the samples were placed between the faces of a compression plate on the top and a compression anvil on the bottom (Instron, cat. no. 2501-107 for the upper plate, 2501-085 for the lower anvil). Compression was carried out to between 7% and 20% strains at a rate of 0.5 mm/min. In most cases, the specimen was unloaded in a controlled manner and the hysteresis recorded. Uniform deformation was assumed. The initial cross-sectional area was used in the following calculations. The compressive strength was defined as the point at which lines from the initial linear region and terminal linear region intersected. The elastic modulus was calculated as the ratio of stress to strain or the slope of the initial linear region of a stress versus strain plot, using the initial cross-sectional area in the calculations.

Determination of Shrinkage

The shrinkage of scaffolds was determined by measuring the diameter and/or thickness of the scaffold with a micrometer. The measurements were taken at several time points during leaching, while the scaffolds were still wet.

Seeding of Scaffolds

The seven batches of disk scaffolds that were evaluated for the cartilage region were screened for their ability to support cellular attachment, cellular colonization, and matrix deposition using dermal fibroblasts as a representative attachment-dependent and matrix-synthesizing cell type. Scaffolds were pre-wetted in ethanol (70%) for 1 minute, disinfected in antibiotic/antimycotic (20× concentration; Gibco, Gaithersburg, Md.) overnight and pre-treated in culture medium (Dulbecco's Modified Eagle medium [DMEM; Gibco], supplemented with bovine calf serum [10%; Hyclone, Logan, Utah], sodium pyruvate [Gibco], non-essential amino acids [Gibco], L-glutamine [Gibco], and antimicrobial agents [Gibco]) overnight. Each disk was seeded for 18-24 hours with 1×10⁶ dermal fibroblasts in 500 μL of culture medium under gentle agitation. The disks were cultured statically in culture medium supplemented with ascorbate (50 μg/mL; Baker, Phillipsburg, N.J.) for 4 weeks in 37° C., 5% CO₂, humidified incubators.

Osteochondral devices were either cultured rotationally by submerging in a tube or top-seeded by pipetting the cells onto the top of the scaffold. Before seeding with chondrocytes, the devices were first pre-wetted in ethanol (100%) for 15-60 minutes. The ethanol was removed by rinsing in PBS three times (5-10 minutes each rinse on a shaker) and the scaffolds were soaked overnight in antibiotic/antimycotic solution to disinfect. The scaffolds were placed in DMEM medium containing 10% fetal bovine serum (FBS, Hyclone) and 25 microgram/mL gentamicin sulfate (GS) (Gibco) for four hours prior to seeding. Scaffolds that were rotationally seeded were placed in a 15-mL conical centrifuge tube that contained 15×10⁶ ovine articular chondrocytes (OAC) from the femoral condyle and filled full with the above medium. The scaffolds were rotated end-over-end overnight in an incubator. For scaffolds that were top-seeded, 15×10⁶ OAC were concentrated in 250 microliter and pipetted on top of the constructs placed in the wells of 6-well plates. The top-seeded scaffolds were left undisturbed for 3.25 hours to allow for the cells to settle and attach to the scaffolds, after which time more medium was added to the wells to prevent dessication. Both sets of scaffolds were cultured statically in 6-well plates for 4 weeks in 37° C., 5% CO₂, and humidified incubators.

Biochemical Analyses

Biochemical analyses were performed at 1, 2, 3 and 4 weeks for the final seven candidate systems and after 4 weeks in culture for the osteochondral scaffolds.

MTT

Estimation of cellular activity and spatial distribution was accomplished using the MTT assay. MTT (3-[4,5-Dimethylthiazol-2-yl]-2.5-diphenyltetrazolium bromide) is a dye that measures cell activity and is taken up by the mitochondria and converted to a blue color for viable and metabolically active cells. Briefly, samples were incubated in MTT solution (0.5 mg/mL in 2% fetal bovine serum culture medium) (Sigma) for 2 hours and rinsed with PBS for 5-10 minutes. The insoluble precipitant was extracted in isopropanol (5 mL) for 24 hours at room temperature, and the optical density (OD) was determined at 540 nm. Linear correlations between OD and cell numbers were previously established.

DNA/cell Number

The total amount of DNA was determined utilizing a Hoechst 33258 dye (Molecular Probes, Eugene, Oreg.) method that was modified for use in a microtiter plate reader. Briefly, samples were digested overnight at 37° C. in papain solution (1 mg/mL in PBS; Sigma) and reacted with Hoechst dye (0.5 microgram/mL) in the dark for 30 minutes at room temperature. After incubation, fluorescence was quantified using a plate reader (Cytofluor®, Persceptive Biosystems, Inc., Framingham, Mass.) and concentrations were determined against a standard curve made from bovine thymus DNA. Cell numbers were calculated using the estimated value for cellular DNA content of 7.7 pg DNA/cell.

GAG

Sulfated glycosaminoglycans (S-GAG) were determined spectrophotometrically by a method adapted for use with a microtiter plate reader. Briefly, aliquots of the papain-digested sample solution (see DNA section above) were mixed with 1,9 Dimethylmethylene blue (DMMB; Aldrich, Milwaukee, Wis.) dye solution and read on a plate reader (Molecular Devices, Sunnyvale, Cali.) with a dual wavelength setting of 540/595 nm. A standard curve was generated using chondroitin-4-sulfate (Sigma) and used to determine the concentration of S-GAG in the samples.

Collagen

Total collagen was indirectly determined spectrophotometrically by the presence of hydroxyproline by a method adapted for use with a microtiter plate reader. Briefly, aliquots of the papain-digested sample solution (see DNA section above) were hydrolyzed with concentrated hydrochloric acid (6N), dried, and resuspended in a sodium phosphate buffer, pH 6.5. The presence of hydroxyproline was detected by an oxidation reaction with chloramine T/P-DAB at 60° C. for 30 minutes. A standard curve was generated using L-hydroxyproline and used to determine the concentration of hydroxyproline in the samples. The calculation of collagen content was based on the estimated percent of hydroxyproline in collagen of 14.3%.

Histology

Histological specimens were fixed in 10% neutral buffered formalin and processed for either paraffin or plastic embedding. Plastic-embedded samples were catalyzed in glycol methacrylate and allowed to polymerize at room temperature for approximately 1 hour. The blocks were sectioned using an automated microtome, and sections (3-4 μm in thickness) were mounted on glass slides. After drying for approximately 1 hour at room temperature, the slides were stained with hematoxylin and eosin or safranin-O to visualize cell and tissue components by light microscopy.

Statistical Methods

One-way analysis of variance (ANOVA), using commercially available statistical software, Sigma Stat, was performed to determine whether significant differences existed between the biochemical results. Post-hoc Tukey testing or Dunn's method (for data sets that failed the normality or equal variance testing) were used for subsequent pairwise comparisons.

Results

Materials Selection for the Cartilage Region

Solvent-cast thin films were qualitatively evaluated over three weeks for rates of degradation and structural integrity to narrow the polymer combinations down to seven final candidates. Films were eliminated if they crumbled or tore easily. In addition, flexible materials were viewed as preferable over rigid materials. At three weeks, the goal was to have the film mostly degraded so films that did not show significant degradation were eliminated. Seven candidate polymer combinations were chosen by this process and were then fabricated into 3-D scaffolds, and tested in vitro for cell attachment and infiltration using dermal fibroblasts as a test cell type.

TABLE 1
Polymer Combinations

| Polymer | Weight | | Weight | |
|---|---|---|---|---|
| Combo | Percent | Polymer | Percent | Polymer |
| 1 | 50% | PLGA(50:50) | 50% | L-PLA I.V. 0.34 dL/g |
| | | I.V. 0.48 dL/g | | |
| 2 | 50% | PLGA(50:50) | 50% | L-PLA I.V. 0.34 dL/g |
| | | I.V. 0.48 dL/g | | |
| 3 | 50% | PLGA(75:25) | 50% | L-PLA I.V. 0.34 dL/g |
| | | I.V. 0.24 dL/g | | |
| 4 | 70% | PLGA(50:50) acid | 30% | L-PLA I.V. 0.99 dL/g |
| | | I.V. 0.18 dL/g | | |
| 5 | 70% | PLGA(50:50) | 30% | L-PLA I.V. 0.99 dL/g |
| | | I.V. 0.48 dL/g | | |
| 6 | 100% | PLGA(50:50) | — | — |
| | | I.V. 0.48 dL/g | | |
| 7 | 100% | PLGA(75:25) | — | — |
| | | I.V. 0.6 dL/g | | |

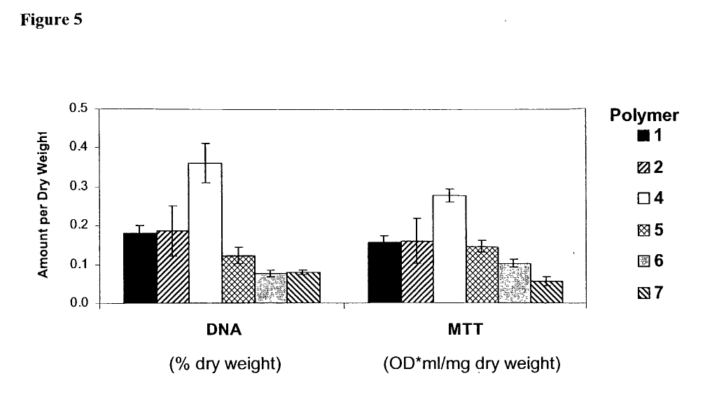

FIG. 5 is a graph of biochemical results of TheriForm™ scaffolds created with polymers 1-7 and cultured statically with dermal fibroblasts for 4 DNA and MTT values were significantly greater for polymer 4 (p<0.05, one-way ANOVA with Tukey post-hoc testing). Bars represent means±standard deviations for n=3, except for polymer 4 (n=2) and the DNA results for polymer 7 (n=2). Analysis of the constructs for MTT and DNA showed the highest levels for polymer combinations 1, 4 and 5 and the lowest for combination 7. Two of the candidates (6 and 7) could not tolerate the residual solvent removal process (i.e., pores collapsed) and were eliminated. One combination (3) was too fragile to be fully tested and was ruled out. Combinations 4 and 6 both deformed significantly (i.e., curled) after four weeks in culture. Gross morphology and histology indicated that candidates 2, 4, 6, and 7 had tissue development primarily on the surface of the device. In contrast, candidates 1 and 5 supported cell attachment and viability, and matrix deposition throughout the cartilage region and maintained the original shape of the scaffold. Candidate 1 was chosen over 5 because 5 contained a higher molecular weight L-PLA that would likely take longer to resorb than was considered desirable.

Mechanical Testing of the Bone Region

A set of scaffolds in which the composition of L-PLGA (85:15), NaCl, and TCP were systematically varied was tested for mechanical properties. The results of some of the mechanical tests are reported in Table 2.

TABLE 2
Tensile and Compressive Testing Data. Averages and standard
deviations for n = 3 or 4.

| Composition | | | Tensile Data | | Compressive Data | |
|---|---|---|---|---|---|---|
| | | L- | Tensile | Elastic | Yield | Elastic |
| NaCl | TCP | PLGA | Strength | Modulus | Strength | Modulus |
| (%) | (%) | (%) | (MPa) | (MPa) | (MPa) | (MPa) |
| 25 | 25 | 50 | 5.7 ± 1.0 | 200 ± 57 | 13.5 ± 0.3 | 233 ± 26 |
| 35 | 15 | 50 | 5.5 ± 0.8 | 233 ± 27 | 13.7 ± 0.8 | 450 ± 79 |
| 35 | 21.7 | 43.3 | 3.3 ± 0.4 | 180 ± 14 | 6.5 ± 0.2 | 184 ± 12 |
| 40 | 15 | 45 | 4.0 ± 0.5 | 183 ± 35 | 7.0 ± 0.9 | 180 ± 50 |
| 55 | 11.25 | 33.75 | 1.6 ± 0.2 | 83 ± 18 | 2.5 ± 0.1 | 54 ± 17 |

| | | | |
|---|---|---|---|
| Cancellous Human | ˜8 | ˜700-1,000 | 10-20 |
| Bone (fresh) [52] | | | |

The general observations were as follows:

1. increasing porosity (or increasing percent of NaCl) decreased the elastic modulus, tensile strength, and strength;

2. increasing polymer content (i.e., increasing polymer/TCP ratio at a constant porosity) increased the strength and elastic moduli;

3. specimens with a higher fraction of TCP tended to exhibit brittle fracture under tension, and samples with a lower fraction of TCP displayed ductile

4. increasing the TCP content decreased the percent elongation to failure.

The bone portion was designed with a lower porosity (55%) than the cartilage region (90%) to give this section more mechanical strength. Choosing a porosity for the bone region required balancing mechanical properties, which are closer to bone at low porosities, and high surface area, which promotes vascularization and bone ingrowth and increases with increasing porosity. An interconnected pore structure was desirable for bone ingrowth and requires a minimum of 32% porosity to be fully interconnected according to percolation theory (assuming a simple cubic lattice). Mechanical properties started to decline around 55% porosity and therefore 55% was chosen as the upper acceptable limit. Current bone repair products such as Interpore-200 and Medpor have porosities in the 50-65% range. Cancellous bone, which is used for autografts and allografts, has a porosity of 50-90%. Thus, 55% was chosen as the porosity of the bone region. Additionally, a large pore size was used (>125 microns) in the bone region to further facilitate mineralized bone ingrowth and mechanical strength. Since in vivo bone ingrowth is a gradual process, unlike in vitro cell seeding which occurs at a given instant in time, the low porosity prevented chondrocyte attachment in the bone region during seeding, as desired, but is anticipated to allow bone ingrowth in vivo. In addition, during bone ingrowth, the porosity will increase with resorption, facilitating bone ingrowth.

Architecture of the Bone Region

In addition to the mechanical properties of the bone portion of the device, the overall outer shape of the device was specifically designed to address several issues. The bone portion was constructed in a cloverleaf shape to specifically:

1. allow the migration of blood and bone marrow-borne tissue forming elements;

2. maximize the surface-area-to-volume ratio to promote bone ingrowth;

3. maximize compressive and torsional strength (to withstand implantation);

4. minimize the amount of polymer (to minimize the cost of device and possible inflammatory response, and promote homogeneous bone formation);

5. be easy to fabricate.

Several different shapes were considered, including a hollow cylinder and a honeycomb structure. Balancing the variables above, the cloverleaf shape was selected as this would provide mechanical rigidity and allow for a reasonable amount of bone integration.

Prevention of Delamination in the Transition Region

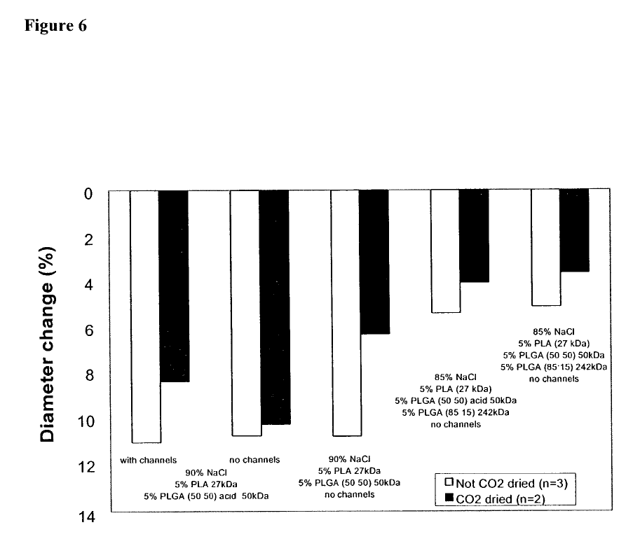

When the first prototype scaffolds were manufactured, it was discovered that during exposure to prolonged leaching (>24 hours), delamination occurred between the cartilage and transition regions. The cause of the delamination was attributed to a significant level of differential shrinkage between these two regions. FIG. 6 is a graph of the amount of shrinkage of scaffolds after leaching for 48 hours. The adjacent transition region was found to shrink 3.8% in diameter compared to 8.3% for the cartilage region. This caused excessive shear stress and may have been responsible for the delamination.

A study was performed to investigate the parameters suspected to cause shrinkage and to improve the structural integrity of the composite scaffolds. Some of the results included:

1. the use of PLGA(50:50) with free acidic side chains increased shrinkage versus regular PLGA(50:50);

2. scaffolds containing 90% NaCl shrank more than those with 85% NaCl;

3. macroscopic channels decreased shrinkage when scaffolds were liquid CO₂ treated;

4. removing residual solvent with liquid CO₂ reduced shrinkage;

Additional results of the study included:

1. scaffolds composed of crystalline L-PLA with an inherent viscosity (I.V.) of 1.1 dL/g and 75% or 90% NaCl shrank less than 2%;

2. shrinkage increased with increasing leaching time;

3. leaching at room temperature reduced shrinkage compared to leaching at 37° C.;

4. shrinkage occurred during the leaching phase and not afterwards during drying.

By using a gradient of materials and porosity to slowly change from one material system to the other, delamination was overcome. It was also found that removing the residual chloroform before leaching reduced shrinkage, since the chloroform can act as a plasticizer. The addition of macroscopic channels slightly decreased shrinkage of CO₂ dried scaffolds, a distinct advantage since the channels enhance cell seeding in the cartilage region.

Final Osteochondral Scaffold Composition and Design

The osteochondral scaffolds consisted of three distinct regions (see Table 3). The bone region was 4.4 mm high and fabricated with 33.75 wt % L-PLGA(85:15) I.V. 1.45 dL/g (Birmingham Polymers Inc., Birmingham, Ala.) milled to 38-150 microns, 11.25 wt % TCP (Sigma) 38-106 microns, and 55 wt % NaCl (Fisher) 125-150 microns. The bone region was shaped as a cloverleaf. The cartilage region was 2 mm tall and fabricated with 5 wt % D,L-PLGA(50:50) I.V. 0.48 dL/g (Boehringer Ingelheim, Germany) and 5 wt % L-PLA I.V. 0.34 dL/g (Birmingham Polymers Inc.), both milled to 63-106 microns, and 90 wt % NaCl that was 106-150 microns. Staggered channels that were approximately 250 microns were incorporated into the cartilage region. The transition region (1.2 mm) consisted of three sections: 65 wt %, 75 wt %, and 85 wt % NaCl with 30 wt %, 15 wt %, and 5 wt % L-PLGA(85:15), respectively. The balance of the transition sections was composed of a 1:1 ratio of D,L-PLGA (50:50) and L-PLA.

TABLE 3
Composition of Osteochondral Scaffold

| | Amount | Size of | PLGA | | PLGA | |
|---|---|---|---|---|---|---|
| | of NaCl | NaCl | (50:50) | PLA | (85:15) | TCP |
| Region | (wt %) | (microns) | (wt %) | (wt %) | (wt %) | (wt %) |
| Cartilage | 90 | 106-150 | 5 | 5 | — | — |
| Transition | 85 | 106-150 | 5 | 5 | 5 | — |
| Transition | 75 | 106-150 | 5 | 5 | 15 | — |
| Transition | 65 | 106-150 | 2.5 | 2.5 | 30 | — |
| Bone | 55 | 125-150 | — | — | 33.75 | 11.25 |

Seeding of the Osteochondral Device-Selective Cell Attachment

Top and rotational seeding were investigated to determine the best method to facilitate chondrocyte attachment and proliferation in the cartilage region and prevent chondrocytes from adhering to the bone region. Chondrocytes preferentially seeded into the cartilage portion of the device and cell attachment to the bone region was minimal.

Sn osteochondral scaffold having staggered channels in the 90% porous cartilage region to facilitate homogeneous seeding has a cloverleaf bone region to promote bone ingrowth in vivo. The bone region is 55% porous. FIG. 7 is a graph of the biochemical results for TheriForm™ osteochondral scaffolds that were seeded with OAC cells by a top or rotational seeding method and cultured statically for 4 weeks. The top seeding method resulted in greater number of cells and S-GAG content in the scaffolds (p<0.001). Collagen content was not statistically different for the two seeding methods and was most likely due to the large standard deviation of the rotational seeded samples.

Although the same number of cells per scaffold were seeded in both methods, the top seeding method resulted in higher cell, S-GAG, and collagen contents than rotational seeding owing to the higher cell concentration with the top-seeded method (in 0.25 mL) compared to the rotational method (in 15 mL).

The chondrocytes seeded and proliferated homogeneously throughout the 2-mm thickness of the cartilage region due to the high porosity and staggered channel design. Histological analysis showed that after 4 weeks in culture, the chondrocytes had populated the cartilage scaffold and deposited an extracellular matrix containing glycoaminoglycans (as detected by safranin-O staining), as has been seen in other tissue engineered cartilage constructs.

The resulting cartilage-bone composite scaffold has two distinct regions (cartilage and bone) composed of different materials, porosity, pore sizes, architectures, and resulting mechanical properties—each specifically optimized for either cartilage or bone. Fabricating a device with two such varying properties without delamination (i.e., splitting apart) was made possible by using a gradient of materials via the TheriForm three-dimensional printing process.

The candidates of polymer combinations for the cartilage region were first screened by qualitatively evaluating the degradation of solvent-cast films in PBS at 37° C. for 3 weeks to select seven candidate polymer combinations. To facilitate cell attachment, proliferation, and matrix deposition, 90% porosity and staggered channels were used in the cartilage region. The remaining candidates were fabricated into scaffolds similar to the cartilage region and cultured with dermal fibroblasts for up to 4 weeks and evaluated by gross morphology, biochemical analyses and histology. From these results, a 1:1 ratio of D,L-PLGA(50:50) I.V. 0.48 dL/g and L-PLA I.V. 0.34 dL/g was selected. The seeding method and extent of matrix deposition was determined with the full osteochondral scaffold design. The best cell seeding method was found to be a top seeding approach.

Results from preliminary mechanical testing of the bone region showed some expected trends. Both the tensile and compressive strengths decreased as the porosity (i.e., void fraction) in the scaffolds increased from 25% to 55%. Likewise, the elastic modulus generally decreased with increasing void fraction. Under ideal conditions, one expects values of the elastic modulus obtained by tensile testing to correspond to the values of the elastic modulus obtained by compression testing. Often, values obtained by compression testing are slightly higher due to friction from the plates. In the samples tested here, it was striking that such agreement was obtained (with the exception of the 35% NaCl: 15% TCP:50% PLGA specimen) between the two different methods. This agreement was especially significant because the orientation of the devices during fabrication was not the same in the samples used for each test. Tensile testing was carried out with samples built so that layers were aligned with the direction of strain, while the compression samples were built so that the layers were aligned normal to the direction of strain. Values for the tensile strength of these devices are comparable to the tensile strength of cancellous bone and values for the compressive strength are within an order of magnitude of the compressive strength of cancellous bone (Table 2). Even though scaffolds generated with porosities lower than 55% were stronger than scaffolds generated with a porosity of 55%, the porosity of the bone region was chosen to be 55% (with a pore size of >125 microns) to balance strength with the potential for in vivo bone ingrowth. The mechanical testing results suggest that the bone region of these scaffolds may have acceptable mechanical properties for in vivo applications as a bone void filler. The compressive properties of the chosen bone region of the scaffold are slightly lower than that of cancellous bone. However, the scaffold will be invaded by new bone and remodeled while the scaffold continually degrades. It is likely that the mechanical strength of the scaffold will significantly increase with bone ingrowth. It is important to note that the properties shown here are for dry samples that had been exposed to aqueous solution only long enough to leach the salt. The mechanical properties at the time of implantation will be somewhat altered due to the aqueous environment, and potentially other factors such as swelling and loss of adhesion between the TCP and polymer particles.

The cloverleaf shape of the bone region was designed to allow adequate contact between the scaffold and surrounding bone in vivo for bone ingrowth but also leaves channels for bone marrow derivatives to contact a large surface area. This design was also created to be able to withstand torsional stress. It is important for the bone portion to be mechanically strong in order to withstand surgical implantation. Furthermore, the bone portion will ideally start to degrade during the bone ingrowth process. In addition to the incorporation of calcium phosphate, other osteoconductive and osteoinductive agents (e.g., BMPs) could be included.

The initial delamination seen between the cartilage and bone regions likely resulted from differential shrinkage of the two regions. It is has been reported that L-PLA has a glass transition temperature (T_{g}) of 57-65° C., and D,L-PLGA (50:50) undergoes a glass transition near 45-55° C. Scaffolds made with a 1:1 ratio of D,L-PLGA(50:50) and L-PLA have a T_{g} of approximately 53° C.

Thus, it is unlikely that the shrinkage occurred due to plastic flow of the amorphous polymer while leaching at 37° C. These results suggest two possibilities: 1) the polymer in the device contains residual elastic strain around the NaCl particles which could be caused partially by collapse of the polymer (e.g., shrinkage of the overall dimensions of the device) when the supporting NaCl is leached out, or 2) the shrinkage was due to hydrostatic pressure.

In this device, a gradient of materials and porosity was used to overcome delamination. Delamination often occurs between regions where the material changes drastically, owing to the different physical properties of the materials (e.g., thermal expansion coefficient, elasticity, etc.) and structure of the regions (i.e., porosity). Using a gradient of materials and architectures, these physical properties were changed gradually, thereby preventing large discontinuities that could result in delamination. Using a gradient of materials was not enough to prevent delamination; it was also necessary to use a porosity gradient. Such gradients were easy to incorporate into the TheriForm process, which builds devices one layer at a time.

The high porosity of the cartilage region (90%) and low porosity of the bone region (55%) allowed the scaffolds to be fully submerged and exposed to chondrocytes during seeding, yet the chondrocytes preferentially attached to the cartilage region as desired. The unique macroscopic staggered channels in the cartilage portion of the device allowed chondrocytes to be seeded in vitro throughout the thickness of the device, not just on the top surface. This uniform seeding is important for rapid, homogeneous cartilage formation since chondrocytes cannot migrate easily over a large (2-mm) distance. Thus, these staggered channels facilitated the direct seeding of chondrocytes into the center of the cartilage portion of the device. In addition, these channels allowed the transport of nutrients to the cells and removal of cellular by-products and polymer degradation by-products away from the cells during culture.

In summary, the TheriForm or three dimensional printing process has permitted the formation of a complex composite suitable as a cartilage-bone tissue engineered scaffold for implantation into articular defects. The versatility of the technology has allowed for a gradient of polymers, and various shapes and internal architectures to be incorporated. The mechanical testing and in vitro production of a cartilaginous matrix in the cartilage region of the scaffolds using chondrocytes indicate that these osteochondral devices have the potential to successfully repair articular defects in vivo. It is anticipated that this technology could be expanded to repair large regions of articular joints, and potentially whole joints for the treatment of osteoarthritis. It is also possible that this technique for making constructs, having a region suitable for one type of tissue adjoining a region suitable for another type of tissue, could also be used for making tissue-growing constructs for the bone-tendon interface and possibly for other tissue-tissue interfaces as well.

Modifications and variations of the foregoing methods and compositions are obvious to those skilled in the art and are intended to be encompassed by the following claims. The references and parent application are specifically incoporated by reference herein.

We claim:

1. A composite medical implant comprising
multiple regions having a different composition, the regions comprising a combination of structure and chemical composition varying from one region to another region to prevent delamination and to promote cell seeding, cell attachment, cell ingrowth or differentiation of cells when implanted into a patient.

2. The implant of claim 1 wherein the implant comprises a curved surface.

3. The implant of claim 2 wherein the implant comprises a curved surface which is curved in more than one orthogonal direction.

4. The implant of claim 2 wherein at least one of the regions comprises a curved boundary with another region.

5. The implant of claim 4 wherein the curved boundary is curved in more than one orthogonal direction.

6. The implant of claim 1 comprising one or more gradients from one region to another region.

7. The implant of claim 6 wherein the gradient is of structure.

8. The implant of claim 6 wherein the gradient is of composition.

9. The implant of claim 7 wherein the structure is porosity.

10. The implant of claim 7 wherein the gradient is of pore size.

11. The implant of claim 1 wherein the implant is a bone-cartilage implant including bone forming and cartilage forming regions comprising
a gradient of materials and porosity between the bone forming and the cartilage forming regions.

12. The implant of claim 11 comprising
a bone forming region having a porosity of approximately 45-65% and
a cartilage forming region having a porosity of approximately 90%.

13. The implant of claim 11 comprising an interconnected region having a minimum of 32% porosity.

14. The implant of claim 11 having a pore size of greater than 100 microns in the bone forming region.

15. The implant of claim 11 wherein the bone forming region comprises a cloverleaf shape.

16. The implant of claim 11 comprising osteoconductive and/or osteoinductive agents.

17. The implant of claim 16 wherein the osteoconductive material is selected from the group consisting of hydroxyapatite, calcium-phosphorus compounds, bone and demineralized bone matrix.

18. The implant of claim 1 formed by solid free form fabrication.

19. The implant of claim 18 formed by three dimensional printing.

20. The implant of claim 1 further comprising one or more agents selected from the group consisting of growth stimulating or differentiating factors and imaging agents.

21. A method to reduce delamination between regions of an implant comprising one or more of the following steps selected from the group consisting of
selecting a polymer optimized to degrade at a controlled rate;
forming a scaffold containing leachable particulate;
forming the scaffold with macroscopic channels;
removing residual solvent; and
leaching at room temperature;
wherein the implant contains regions having a composition and porosity selected to avoid delamination due to shrinkage.

22. The method of claim 21 comprising removing residual solvent using liquid or supercritical carbon dioxide.

23. A method of repairing or replacing tissue comprising implanting into a patient a composite medical implant comprising multiple regions having a different composition, the regions comprising a combination of structure and chemical composition varying from one region to another region to prevent delamination and to promote cell seeding, cell attachment, cell ingrowth or differentiation of cells when implanted into a patient.

24. The method of claim 23 wherein the tissue is bone.

25. The method of claim 23 wherein the tissue is bone and cartilage.

26. The method of claim 23 wherein the implant is seeded by placing the implant in a suspension of cells wherein the cells attach to sites on the implant based on the porosity at the sites.

27. The method of claim 23 wherein the porosity is at least 85%.

28. The method of claim 23 wherein the pores are 125 microns or greater for seeding of cells for forming bone.

29. A method of making a composite implant, comprising:
depositing a layer of powder comprising non-leaching solid particles and porogen particles, wherein the composition of the non-leaching solid particles or the proportion between non-leaching solid particles and the porogen particles can vary from place to place within the layer;
depositing onto the layer of powder in selected places a binder liquid suitable to bind the particles together;
allowing or causing the binder liquid to at least partially dry;
repeating the above steps as many times as desired to form a three-dimensional object;
separating the three-dimensional object from unbound powder particles; and
leaching the porogen from the resulting object by dissolving the porogen in a solvent which dissolves the porogen but not the non-leaching solid particles.

30. The method of claim 29 wherein the non-leaching solid particles comprise one or more substances selected from the group consisting of: resorbable polymers, nonresorbable polymers, hydroxyapatite, tricalcium phosphate, other calcium-phosphorus compounds, other ceramics, bone particles, and demineralized bone matrix.

31. The method of claim 29 wherein the porogen is soluble in water.

32. The method of claim 31 wherein the porogen comprises a water soluble salt or sugar.

33. The method of claim 29 wherein the layer of powder is deposited by dispensing a slurry or suspension comprising the non-leaching solid particles and the porogen particles in a carrier liquid, the proportion between the solid non-leaching particles and the porogen particles in the suspension being variable from one place to another in the layer of powder.

34. The method of claim 29 wherein the depositing is performed by a single dispenser.

35. The method of claim 29 wherein the depositing is performed by more than one dispenser.

36. The method of claim 29 wherein the dispensing is performed by one or more piezoelectric dispensers.

37. The method of claim 29 wherein the dispensing is performed by one or more microvalve dispensers.

38. The method of claim 29 wherein the layer of powder comprises at least two regions, each region having its own composition of powder.

39. The method of claim 29 comprising spreading a non-uniform composition powder within a layer using a roller to deposit the powder.

40. The method of claim 29 wherein the powder is deposited as a slurry or suspension.

41. The method of claim 40 comprising depositing one or more slurries or suspensions at least one of which comprises non-leaching solid particles and the porogen particles.
